(19) United States
(12) Patent Application Publication
(10) Pub. No.: US 2022/0176164 A1
(43) Pub. Date: Jun. 9, 2022

(54) TREATMENT DEVICES FOR ACOUSTIC WAVE STIMULATION

(71) Applicant: Creaholic S.A., Biel (CH)

(72) Inventors: Marcel Aeschlimann, Ligerz (CH); Naomi Bitmead, Bremgarten bei Bern (CH); Carole Chapelat, Biel (CH); Samuel Malzach, Evilard (CH); Martin Sigrist, Bern (CH); Yann Schaeffer, Lichtensteig (CH); Laurent Torriani, Lamboing (CH); Loïc Sottas, Lausanne (CH)

(21) Appl. No.: 17/441,860

(22) PCT Filed: Mar. 25, 2020
(86) PCT No.: PCT/EP2020/058442

§ 371 Date: Sep. 22, 2021

(30) Foreign Application Priority Data
Mar. 25, 2019 (CH) 00395/19

(51) Int. Cl. A61N 7/00; A61B 90/00

(52) U.S. Cl. CPC A61N 7/00; A61B 90/36; A61B 2090/3937; A61N 2007/0078; A61B 90/39

(57) ABSTRACT

The invention relates to the field of cell stimulation by mechanical energy, in particular by acoustic energy, for example stimulation by ultrasound, such as focused ultrasound (FUS). It relates to treatment devices and components thereof as well as methods for setting up or producing such devices or components. One purpose of the invention is to make devices for cell stimulation by mechanical energy more user friendly and applicable for a wider public and for additional applications. A treatment device for acoustic wave stimulation to a body portion is portable and includes a carrier element, at least one transducer and communication device configured to receive operating parameters for the at least one transducer from a remote computerized device. A method for setting up a treatment device for acoustic wave stimulation includes receiving operating parameters for the at least one transducer from a remote computerized device.

### BACKGROUND OF THE INVENTION

### Field of the Invention

The invention relates to the field of cell stimulation by mechanical energy, in particular by acoustic energy, for example stimulation by ultrasound, such as focused ultrasound (FUS). It relates to devices for acoustic wave stimulation to a body portion and methods for setting up such devices.

### Description of Related Art

US 2017/0291044 A1 discloses an ultrasound deep brain stimulation method and an ultrasound deep brain system. The system disclosed includes an ultrasound transducer array and a head fixing and locating device provided in a magnetic resonance imaging (MRI) system. The system of US 2017/0291044 A1 includes further a medical imaging equipment for photographing a head image, wherein a head 3D digital model according to the head image is generated in a step subsequent to photographing the head image. This complex and large-scale system is used for carrying out the ultrasound deep brain method disclosed in US 2017/0291044 A1 and for fine tuning and controlling a stimulation based on this method.

The ultrasound deep brain method disclosed in US 2017/0291044 A1 is a time reversal method and includes the steps of creating the head 3D digital model from the head image, creating a 3D digital model of an ultrasound transducer array, and determining a voltage signal to be applied to the “real” ultrasound transducer array used to generate a specific ultrasound field in the brain by placing virtual sound sources at one or more positions that need to be focused, simulating propagation behaviour of ultrasound wave emitted from the virtual sound sources in the head 3D digital model, and simulating a voltage signal of the ultrasound transducer array when the ultrasound wave is propagated to a virtual spatial position where the ultrasound transducer array is located. In an embodiment, a model of the skull is printed, “real” sound sources are placed at one or more positions, and the voltage generated by an ultrasound transducer array arranged on the printed skull is measured in order to determine the voltage signal to be applied to the ultrasound transducer array.

US 2012/0029352 A1 aims to provide an ultrasound device that enables the use of ultrasound in previously inaccessible medical fields, in particular for tumor treatment. The ultrasound device is a device for a specialised medical facility and it can be operated by a specialist, only. The teaching of US 2012/0029352 A1 aims in particular to overcome the issue that the curvature of the body portion to be treated, for example the head, are specific to the patient and therefore is individual. The ultrasound device disclosed in US 2012/0029352 A1 has a transducer formed by multiple transducer elements that are housed in a flexible carrier, a measurement device and a control device. The measurement device is fashioned to determine from a test series the distances and the rotations of the transducer relative to a reference point, and hence to determine the patient-specific position of the transducer elements, wherein the individual transducer elements emit sound waves during the test series. The control device is able to determine the sound intromission angles and the focus points required for the treatment of a location, e.g., a point of a tumor, specified by a physician, wherein the control device uses the patient-specific position of the transducer elements provided by the measurement device. In embodiments, the control device can be fashioned to vary the sound intromission angle and/or the focus point dependent on the application. In an embodiment, the determination of distances and rotations of the individual transducer elements can ensue from older date of the patient. US 2012/0029352 A1 discloses further a related method for determining an activation for the ultrasound device.

EP 2085119 A1 discloses a low-energy ultrasonic therapeutic device that is capable of multipoint transmitting and that has a high use efficiency of ultrasonic energy, this means an increased therapeutic effect in combination with a reduced risk for injuring a healthy area at a given ultrasonic energy. The device includes a substrate, on which an ultrasonic transducer array is arranged, wherein each of the ultrasonic transducers of the ultrasonic transducer array can independently focus. Thereby, unwanted interference among the ultrasonic transducers can be avoided.

US 2018/0130457 A1 discloses a deformable ultrasound device including an array of a plurality of ultrasound transducer elements. Therefore, the device includes a carrier with an actuator arrangement of a material having an adjustable shape in response to an electromagnetic stimulus. The actuator arrangement allows for a change of the orientation of the ultrasound transducer elements and hence facilitates beam shaping and/or body contour matching of the device.

US 2006/0241522 A1 relates to medical ultrasound devices and methods for stimulating angiogenesis and/or relieving ischemia in a human or veterinary patient. The device includes a substrate and multiple, individual transducer elements disposed on the substrate. The device is for use by a specialist and enables individual control over each of the ultrasound transducer elements to provide the most effective individualized treatment to a patient.

The demands on ultrasonic devices that are configured for imaging, for example as disclosed in US 2009/0024034 A1, are different from the demands on ultrasonic devices used for stimulation. Hence, prior art related to ultrasonic devices for imaging is of limited relevance for the present invention.

State of the art methods for cell stimulation by acoustic waves as disclosed in US 2017/0291044 A1, US 2012/0029352 A1, EP 2085119 A1, US 2018/0130457 A1 and US 2006/0241522 A1 have various drawbacks. In particular, the devices used for stimulation are complex, expensive and large-scale. This also means that a stimulation by acoustic waves can only be done in a specialized and accordingly equipped medical facility but not outside such specialized medical facility. Further, the drawbacks prevent acoustic wave stimulation form being used for medical cases that are often rated as less severe, such as medical cases related to muscular tension, migraine, tension/relaxation, learning problems, sleep problems, etc. Even further, the components of the system used to carry out state of the art methods for cell stimulation by acoustic waves have a very specific design that can be adapted to medical cases in a limited manner only and have a user-friendliness that is very limited.

In other words, state-of-the-art devices are too complex, too expensive and too limited in design and realization to be used by a wider public and/or for a wide range of applications, this means for a wide range of medical and non-medical cases.

Applications in the fields of brain therapy, trauma treatment (e.g., bone stimulation, muscle stimulation), pain treatment (e.g., chronic pain, back pain, migraine) or drug delivery (e.g., localized drug delivery, gene therapy) are examples of applications related rather to medical cases. Applications in the fields of wellbeing (e.g., relaxation, less severe cases of insomnia, migraine and muscular tension etc) or learning are examples of applications related rather to non-medical cases.

One important issue in the field of cell stimulation by mechanical energy, in particular acoustic energy, such as (focused) ultrasound energy, is that the parameter space to be considered is huge even if the device is configured for a single application, this means a single medical or non-medical indication, only. Some of the mentioned drawbacks base on this issue and this issue is also a reason why ultrasonic devices used for stimulation are provided to specialists (e.g., practitioners) in the first instance and hence are not individualized, this means designed for a specific body portion of a defined (given) individuum. However, it can be difficult even for specialists to handle the parameter space and to identify and apply the parameters most suitable for a medical or non-medical indication, in particular if the device and/or the specialist offers the treatment of a plurality of indications (applications).

It is important for the success of the stimulation of a body portion that a treatment device used for the stimulation is configured (set-up) in a manner that leads to a specific acoustic wave stimulation at at least one specific point in the body portion to be stimulated. In other words, an acoustic field needs to be generated at a point in the body portion to be stimulated, wherein the position of the point in the body portion to be stimulated and the characteristics of the acoustic field at that point depend on the application.

The huge parameter space is established at least by:
The geometric property of the body portion to be stimulated, in particular its shape, for example its shape at the region at which the stimulation is applied.
The arrangement of the at least one transducer used for generating the stimulation. The arrangement may include the position and orientation of the transducer relative to the body portion and/or relative to each other if more than one transducer is used, for example. It can further include the contact pressure between the transducer and the body portion, for example.
The operating parameters of the transducer(s). Frequency, intensity, pulse duration, pulse length, pulse repetition rate are examples of operating parameters.
The type of transducers used.
The interaction between transducer and body portion.
The body portion to be stimulated, this means the tissues of which it is made, the arrangement of these tissues, the characteristic (in particular mechanical properties) of the tissues, for example.

It goes without saying that the parameter space is multiplied by any additional application (medical or non-medical indication) that can be addressed by a device.

For example, it is important to choose a number and arrangement of transducers as well as operation parameters that fit best for a specific treatment. However, this choice bases on expert knowledge in state-of-the-art devices and methods.

For example, it is important to consider geometric properties of the body portion to be stimulated when configuring (setting up) a system or treatment device that generates an acoustic wave stimulation by arranging at least one transducer on the surface of the body portion. Otherwise, it would not be possible to meet the requirement of a specific acoustic wave stimulation at at least one specific point in the body portion to be treated.

Some of the state-of-the-art systems and devices, for example as disclosed in US 2017/0291044 A1, use complex and large-scale medical imaging equipment, such as an MRI system or a Computer Tomography (CT) system, to determine geometric properties of the body portion to be stimulated.

Some of the state-of-the-art systems and devices, for example as disclosed in US 2017/0291044 A1, use complex and large-scale medical equipment, such as a functional magnetic resonance imaging (fMRI) system, to control and adjust the configuration (set up) of the system or treatment device used for stimulation.

### SUMMARY OF THE INVENTION

It is an object of the invention to overcome drawbacks of state-of-the-art devices and methods.

In particular, it is an object of the invention to provide a treatment device for acoustic wave stimulation that is more user friendly. In particular, the device can be more user friendly by supporting the user to handle the huge parameter space related to acoustic wave stimulation.

For example, it is an object of the invention to provide a treatment device for acoustic wave stimulation that can be applied for one or various applications and by the wider public. The applications can be medical applications and/or applications in the field of well-being.

In particular, it is an object of the invention to provide a portable treatment device that allows for acoustic wave stimulation without need for complex and large-scale medical equipment.

It is a further object of the invention to provide a method for setting up a device for acoustic wave stimulation, in particular, but not exclusively, a device for acoustic wave stimulation that can be applied for one or various applications and by the wider public. The applications can be medical applications and/or applications in the field of well-being.

In particular, it is an object of the invention to provide a method for setting up a device for acoustic wave stimulation that allows for acoustic wave stimulation without need for complex and large-scale medical equipment.

A provider for cell stimulation by mechanical energy for medical cases, such as a specialist for cell stimulation by medical energy or a practitioner, a provider for cell stimulation by mechanical energy for non-medical cases, such as institutions in the field of well-being or sport, and a user of the treatment device that uses the device for himself or for a person of his social environment are examples of potential users of the treatment device and/or the responsible person for carrying out the methods or steps of the methods.

Independent of the embodiments of the device and the methods, the following terms have the following meaning if not stated explicitly otherwise:

A “carrier element” is a carrier structure (a supporting element, a supporting structure) for the at least one transducer (and potentially other elements and means). In other words, it is an element that extends in two dimensions at least, wherein the at least one transducer (and potentially other elements and means) can be arranged on it, in particular fixed on it.

An object, such as a set of treatment parameters or a body portion, is “subject specific” if it is related to an individual (human or animal) subject, such as a patient. For example, measures can have been taken to make sure that the object is related or adapted to an individual patient.

A “set of treatment parameters for acoustic wave stimulation” (in short “treatment parameters”) include operating parameters of the transducer(s) used to generate an acoustic wave field in the body portion to be stimulated and information concerning the arrangement of the transducer(s) relative to the body portion. The latter can include information concerning at least one of an orientation of the transducer(s) with respect to the body portion, a contact pressure between the transducer(s) and the body portion, and the type of transducer to be used, for example. The orientation of a transducer can be given by the angle between an axis, in particular an oscillation axis, of the transducer and the surface normal of the body portion at the position at which the transducer is arranged, for example. Frequency, intensity, pulse duration, pulse length, pulse repetition rate are examples of operating parameters.

Methods for determining, validating and/or adjusting the set of treatment parameters for acoustic wave stimulation are discussed in detail in the application entitled “Treatment parameters for acoustic wave stimulation” (CH application number 00394/19) filed by the same applicant at the same date as the present application. The disclosure of this application is hereby incorporated by reference.

In many embodiments, the “acoustic wave” is an ultrasound wave (in short ultrasound).

A “body portion” can be any body portion, for example the head, the neck, an extremity or a part thereof.

A “library” is an electronic library, this means an electronic database, if not stated otherwise.

A treatment device according to the invention is portable and includes a carrier element, at least one transducer and communication means, in particular a communication unit, configured to receive operating parameters for the at least one transducer from a remote computerized device.

The operating parameters received can include any operating parameter needed for a given stimulation on the body portion to be stimulated.

The operating parameters received can be application- and subject-specific.

The operating parameters received may have been determined by a practitioner or by a computer-implemented method configured to select the operating parameters from a plurality of operating parameters that are stored in a library, for example. Details concerning the operating parameters received are given below, in particular with respect to the method for setting-up the treatment device.

The operating parameters or—as the case may be—the set of treatment parameters received can be the operating parameters of the set of treatment parameters (the set of treatment parameters) that is determined in the application entitled “Treatment parameters for acoustic wave stimulation” (CH application number 00394/19) filed by the same applicant, in particular as disclosed on page 5/line 12 to page 25/line 11. In summary, the operating parameters (the set of treatment parameters) received can be subject specific and can be determined by a method including a step of selecting a set of operating parameters (a set of treatment parameters) from at least two different sets of operating parameters (to different sets of treatment parameters), wherein the step of selecting is made prior to a step of applying any set of treatment parameters to a body portion. The method includes further a step of generating subject specific data, wherein the step of generating subject specific data includes measuring a geometric property of a body portion, and a step of determining a target field distribution in the body portion, wherein the step of determining a target field distribution includes selection of an application. Optionally, at least one of the following may apply:
The step of selecting a set of treatment parameters may include a comparison of a first expected field distribution related to a first set of treatment parameters with the target field distribution and a comparison of a second expected field distribution related to a second set of treatment parameters with the target field distribution.
The step of generating subject specific data includes the substeps of adding tissue characteristics of the body portion to the measured geometric property and of generating a digital (virtual) 3D model of the body portion based on the measured geometric property and on the added tissue characteristics. The digital 3D model may then be used to approximate optimized operating parameters (set of treatment parameters) in an iterative manner.
A step of transmitting the subject specific data to an electronic library, wherein the electronic library is configured to carry out the step of determining a target field distribution and the step of selecting a set of treatment parameters.

The operating parameters or—as the case may be—the set of treatment parameters may be validated by the method for validating as disclosed on page 26/line 4 to page 33/line 13 and/or adjusted as disclosed on page 33/line 19 to page 34/line 11 of the above mentioned application entitled “Treatment parameters for acoustic wave stimulation”. The teaching of the application is herewith incorporated by reference.

In particular, the operating parameters (set of treatment parameters) may be validated before or after being received by the treatment device by a method including a step of generating a subject specific 3D model of the body portion to be stimulated (treated) and a step of determining a difference between the target field distribution and a field distribution determined in the subject specific 3D model. The subject specific 3D model may be a physical (“real”, non-virtual) 3D model or a digital (virtual) 3D model and the field distribution determined by be a measured or calculated field distribution.

The operating parameters (set of treatment parameters) may be adjusted before or after being received by the treatment device by a method including a step of reducing the difference between the target field distribution and the field distribution determined in the subject specific 3D model, in particular if said difference is larger than a preset value.

The method of validating and/or of adjusting as disclosed in the application and as summarized above, may be part of any embodiment of the method for setting-up the treatment device as disclosed below.

In a preferred embodiment, the treatment device itself does not include a computerized device to determine any parameter of the set of treatment parameters. However, the treatment device may include a controller configured to transform the operating parameters received into commands for the transducer(s). The treatment device may also include a computerized device that is capable to execute any computer implemented method disclosed with respect to the functionality of the treatment device in any embodiment.

In other words, the parameter space that has to be handled by the user of the treatment device is reduced by the device being configured to receive the operating parameters required for a treatment from or via the remote computerized device.

In embodiments, at least one of the following aspects is realized in the treatment device for reducing the parameter space:
According to a first aspect the treatment device includes means for determining a geometric property of the body portion when the carrier element is fastened on the body portion, wherein a characteristic of the means for determining a geometric property depends on at least one of the shape of the carrier element and a deformation of the carrier element.
In embodiments according to the first aspect, the treatment device is configured to transmit the geometric property of the body portion to the computerized device. In a method for setting-up a treatment device according to these embodiments, the operating parameters received may depend on the geometric property transmitted.
Embodiments of the first aspect of the invention are in particular suitable for treatment devices that influence the body portion when fastened to the body portion. For example, the shape of the body portion and/or the density of the body portion is influenced.
However, the invention also concerns treatment devices for acoustic wave stimulation of a body portion that do not influence the body portion when fastened to it.
A treatment device according to a second aspect allows for the determination of a position of the at least one transducer relative to the body portion when the carrier element is fastened on the body portion.
In embodiments according to the second aspect, the treatment device is configured to transmit the position of the at least one transducer relative to the body portion to the computerized device. In a method for setting-up a treatment device according to these embodiments, the operating parameters received may depend on the position transmitted.
A treatment device according to a third aspect has a carrier element that has a shape that is specific to the body portion of an individual subject.
The carrier element can have the shape that is specific to the body portion of the individual subject because the shape of the body portion has been determined prior to manufacturing the carrier element, for example by use of a professional medical imaging equipment, a less sophisticated imaging equipment, such as a commercially available 3D scanner, or can by use of routines that are able to approximate the shape of the body portion from a plurality of images of the body portion. The manner of determining the shape of the body portion depends usually from the application.
In many embodiments, the carrier element is manufactured by a specialized manufacturer. However, the carrier element may be manufactured by a platform owned by the end-user, a distributor or a practitioner, for example.

A further aspect that may present instead of or in addition to at least one of the first to third aspect concerns the reduction of the parameter space to be handled by the operator of the treatment device by generating a digital (virtual) 3D model of the body portion to be treated, in particular as disclosed in the application entitled “Treatment parameters for acoustic wave stimulation” (CH application number 00394/19) filed by the same applicant and summarized above. In this context, in particular page 17/line 8 to page 20/line 13 and page 21/line 21 to page 22/line 12 as well as the validation and/or adjustment to which is referenced above are to be considered.

The further aspect concerning the generation of a digital 3D model may be in particular advantageous in a method for setting up the treatment device, but also in a method for manufacturing the carrier element or the treatment device. Any data needed for generating the digital 3D model can be generated for example by a practitioner in a medical facility, for example when determining the shape of the body portion to be treated.

One can envisage embodiments of a treatment device and related methods in which at least one of the first, second, third and further aspect is present without including the communication means (communication unit) configured to receive operating parameters for the at least one transducer from a remote computerized device. In this case, the operating parameters or even the set of treatment parameters may be determined by a computerized device of the treatment device, for example.

In particular embodiments according to the first aspect of the invention can include a carrier element that is elastic or includes an elastic element and that is fastened on the body portion by a force generated by the elastic deformation of the carrier element or its elastic element when arranged to the body portion.

However, the force acts on the body portion and may deform it—in particular in the region of the at least one transducer and/or a fixation element for the at least one transducer. Hence, there is need to determine a geometric property of the body portion after the arrangement of the carrier element.

However, in an embodiment the carrier element can include rigid material that is formed according to a portion of the body portion to be treated, at least. For example, the carrier element includes a surface region that is the negative a surface region of the body portion to be treated. The rigid material can be formed according to a specific body portion in a manner that is not specific to a given individual.

The rigid material can be formed according to the shape of a specific body portion of a given individual.

In such embodiments, fastening of the carrier element on the body portion does not influence the properties of the body portion. Hence, means for determining a geometric property of the body portion when the carrier element is fastened on the body portion is not essential for such embodiments. Further, some geometric properties of the body portion can be known, for example because the shape of the body portion was needed to provide the carrier element that includes rigid material that is formed according at least to a portion of the body portion to be treated.

However, there is need to validate the position of the at least one transducer relative to the body portion when the carrier element is fastened to the body portion in many embodiments, because the relative position of the carrier element and hence of the transducer may vary. Further, the position and orientation of the at least one transducer may be different for different applications.

In an embodiment, the treatment device includes the means for determining a geometric property of the body portion when the carrier element is fastened on the body portion and it allows the determination of a position of the at least one transducer relative to the body portion when the carrier element is fastened on the body portion is a reasonable embodiment of a treatment device.

For example, the geometric property determined can be an input for determining a set of treatment parameters and the observed position of the at least one transducer relative to the body portion can be a validation of the position of the at least one transducer being a target position or it can be a further input for determining the set of treatment parameters.

Generally, a target position of a transducer is a position of the transducer relative to the body portion, said position being required to generate the target field distribution in the body portion when the set of treatment parameter is applied. In many embodiments, the target position(s) is/are part of the set of treatment parameters.

The at least one transducer is a transducer capable to stimulate the body portion by an acoustic wave, such as ultrasound, when arranged on the body portion.

The geometric property determined can be used for at least one of determining a set of treatment parameters, validating a set of treatment parameters, and adjusting a set of treatment parameters.

The treatment device is capable to implement a set of treatment parameters, in particular a subject specific set of treatment parameters.

In embodiments, the means for determining a geometric property of the body portion include means for determining a shape of the body portion to be stimulated.

The means for determining a geometric property of the body portion can consist of means for determining the shape of the body portion to be stimulated

In other words, the geometric property determined can include the shape of the body portion to be stimulated or the geometric property is the shape of the body portion to be stimulated.

However, a geometric property that is different to the shape of the body portion to be stimulated can be determined by the means in addition or alternatively. For example, the shape of a portion of the body portion, for example the shape of a bone, such as the skull or a long bone, or the arrangement of muscles, body fat, etc., can be determined. This can be done for example by the means including a second and potentially a third kind of transducer, as described below.

Alternatively or in addition, the geometric property that is different to the shape of the body portion can be determined by use of a medical imaging system, such as a CT system or a MM system.

It is an advantage of the treatment device that includes the means for determining a geometric property that it can be made sure by determining a geometric property (of the body portion to be stimulated) by the treatment device and when the carrier element is arranged on the body portion that the at least one transducer has a known position relative to the body portion when the at least one transducer is fixed at a known position at the treatment device.

In other words, there is no need for any additional medical imaging system if the treatment device is configured for determining a geometric property of the body portion when fastened on the body portion. This is because it becomes possible to relate a position on the treatment device fastened to the body portion to a position relative to the body portion.

There is no need that an equipment for determining the characteristic of the means for determining a geometric property is an integral part of the treatment device. For example, the equipment for determining can be a handheld device owned by potential users anyway, such as a cell phone, or delivered as a separate part of the treatment device.

A 3D scanner can be the equipment for determining the characteristic of the means for determining a geometric property or it can be an equipment for determining the shape of at least one of the body portion, the carrier element mounted on the body portion, and the position of the at least one transducer mounted to the carrier element when the carrier element is mounted on the body portion.

The 3D scanner can be a portable 3D scanner.

The treatment device can be designed to determine the geometric property of the body portion from determining a geometric property of the treatment device.

In an embodiment, the carrier element includes the means for determining a geometric property of the body portion.

The carrier element can be made from a material that is elastic. In particular, it can be elastic in a manner that the carrier element sits tight on the body portion when arranged on it.

Alternatively, the carrier element can be made from a rigid material and optionally include a flexible or pivoting part that can be used for fastening or tightening, for example.

The carrier element can have a constant thickness when in a relaxed state, at least. The carrier element can be thin in the meaning that a change of thickness due to elongation (stretching) can be neglected when determining the geometric property of the body portion from the geometric property of the treatment device.

The treatment device can be portable for the subject due to its size and weight. The size of the treatment device can be essentially the size of the carrier element, except wires and elements integrated in or connected to the carrier element, such as the at least one transducer or the optional sensors, emitters, transducers of the second kind (discussed below) etc.

The size of the treatment device (the carrier element) can be essentially in the order of the surface area of the largest body portion to be stimulated in the envisaged applications. For example, the size of the treatment device (carrier element) can be adapted to the size of the head, the neck, the shoulder-neck area, the upper arm, the forearm, the tight, the lower leg and the foot.

It can also be envisaged to provide treatment devices for specific applications and or a group of users. Therefore, the treatment devices, in particular the carrier element and/or the transducer type, can differ in size and potentially in shape and fastening elements, for example.

For example, one treatment device can be adapted to the size and shape of the head. Another one can be adapted to the size and shape of extremities or of an extremity. Another one can be adapted to the torso or portions thereof, such as the chest, the back, the pelvis, the shoulder or the shoulder-neck area.

The size of the treatment device (the carrier element) can be adapted to the largest of the body portions for which the treatment device is adapted. Generally, the largest body portion is the body portion having the largest surface area on with the treatment device has to be arranged during stimulation.

For example, the carrier element that defines essentially the size of the treatment device has a surface area of less than 2.25 m², for example less than 2 m², 1.5 m² or 1 m². A surface area of less than 1 m² can be sufficient for most applications.

However, the treatment device (carrier element) can be designed for a sequential treatment of the body portion. In this case, the size of the treatment device can have a surface area that is smaller than a surface area given by the body portion.

The carrier material can be made of a fabric (textile). The fabric can be a synthetic fabric, a non-synthetic fabric or a combination thereof, for example. Hence, the thickness of the carrier element can be below 2 cm, for example below 1 cm or below 0.5 cm, for many embodiments.

The carrier material can include or consist of plastic, for example rigid plastic.

The carrier material can include or consist of light-weight material.

The carrier material can include or consist of material being compatible with skin-contact or wound contact.

The carrier element can be moulded or 3D printed.

The treatment device can be wearable, this means it can be arranged on and fixed to the body portion, for example by tightening around the body portion. For example, the carrier element can be a cuff, a hood or any other article of clothing that can be arranged around a body portion. The carrier element can include elements that are “plaster cast”-like—however without being made from plaster in many embodiments.

The weight of the treatment device is essentially given by the carrier element, the transducer(s) arranged on it and optional elements, such as sensors, emitters, second kind of transducers (see below) etc. potentially arranged on it. The weight of the treatment device is such that an adult can carry and/or wear it. For example, the weight of the treatment device is less than 10 kg, for example less than 5 kg or less than 2 kg.

The treatment device can include portions that are hollow in order to reduce its weight.

The carrier element can include a fastening element arranged to fasten the carrier element on the body portion, in particular to fasten the carrier element to the body portion in a tight manner.

In an embodiment, in particular in an embodiment in which the means for determining a geometric property of the body portion to be stimulated are means for determining the shape of the body portion to be stimulated, the means can include at least one of:
A marker arrangement, wherein the marker arrangement is arranged on the carrier element and wherein the marker arrangement changes its appearance in dependence of the shape of the carrier element.
The marker arrangement can include a plurality of elements, for example positioning points and/or lines.
The positioning points and/or lines can be detectable by an optical system, for example a camera.
If the marker arrangement includes positioning points, the distances between the positioning points determined from a picture can be indicative for the appearance of the marker arrangement, for example. In other words, the characteristic that changes can be a distance (or rather a plurality of distances), for example.
If the marker arrangement includes lines, the shapes, the lengths and the relative arrangement of the lines, said shape, lengths and relative arrangement being determined from a picture, can be indicative for the appearance of the marker arrangement, for example. In other words, the characteristic that changes is at least one of a shape, a length and a relative arrangement of a line, for example.
For example, the positioning points and/or the lines can form a grid on the carrier element, wherein the appearance of the grid depends on the shape of the carrier element.
The positioning points and/or lines can be arranged on a surface of the carrier element that is an outer surface of the carrier element when the carrier element is fastened on the body portion.
The marker arrangement can be arranged on the carrier element in a manner that a program can calculate the shape of the carrier element based on at least one picture taken from the carrier element when fastened on the body portion.
The calculation of the shape can further be based on information of the marker arrangement, for example the arrangement of the positioning points and/or lines, said information can be stored in a library that is in communication with a computerized device, wherein the program is carried out by the computerized device, or it can be extracted from the at least one or another picture.
For example, the marker arrangement can include elements including the information needed for calculating the shape of the carrier element. QR-code or Bar-code are examples of such elements. Alternatively or in addition, said information can be present in elements of lengths and orientation that are specific for the marker arrangement and/or its arrangement of the positioning points and/or lines.
The treatment device can include an element that identifies the treatment device. This can be used to get further information from the library.
A plurality of sensor, wherein the sensors are arranged on the carrier element and wherein an output of the sensors depends on the arrangement of the sensors on the carrier element, this means on the arrangement of the sensors in the 3D space. Distance sensors are an example of sensors capable to be used in means for detecting a geometric property of the body portion, in particular in means for detecting the shape of the body portion to be stimulated. In this case, the characteristic is the distance between the sensors. The distances can be an output of the sensors. Position sensors are another example of sensors capable to be used in means for detecting a geometric property of the body portion, in particular in means for detecting the shape of the body portion to be stimulated. In this case, the characteristic is the position of the sensors in space. The positions of the sensors can be an output of the sensors.
A plurality of emitters, wherein the emitters are arranged on the carrier element and wherein an output of the emitters, this means an emitted signal, that is detectable at a detection point depends on the relative arrangement of the emitters.
For example, all emitters of the plurality of emitters can emit a signal of a given intensity, wherein the intensity detected at the detection point depends from the distance between the emitter and the detection point.
The treatment device can include the detection point.
There can be a plurality (this means at least two) detection points.
The detection point(s) can be an integral part of the treatment device or it/they can be comprised in separate element, such as the handheld device.

If a camera is used for determining a geometric property, the camera or a related computerized device can be configured to recognize a body portion from a taken picture, for example by comparing a body shape to a list of body shapes. For each body portion that the camera or related computerized device is able to recognize, some specific points can be predefined and stored in the camera or computerized device, for example. The camera or computerized device can be configured to identify the predefined points. The camera or computerized device can be configured to calculate the true dimensions of the body portion or to approximate the true shape of the body portion from the predefined points. For example, the predefined points identified on the picture can be set in relation with the related points in a pre-registered picture of the same body part. The true dimension or an approximation of the body part, for example, can then be determined from a difference observed between the predefined points identified on the picture and the related points in the pre-registered picture of the same body part.

However, an equipment different from a camera can be used for determining a geometric property, such as CT-system, an MM-system or a 3D-scanner.

One can also envisage to use a more sophisticated equipment, for example a CT-system, a MRI-system or a 3D-scanner, in an initial step of setting up or manufacture the treatment device and to use another equipment, for example a camera, such as the camera of a cell phone, for a subsequent step of setting up and/or validating the treatment device.

The at least one transducer can be firmly attached to the carrier element. This means, the at least one transducer can be attached to the carrier element in a manner that it cannot be removed by the user.

In embodiments including a plurality of transducers, all transducers—at least all transducers capable to stimulate the body portion by an acoustic wave when arranged on the body portion—can be firmly attached to the carrier element.

In embodiments including a plurality of transducers, the treatment device can include at least one array of transducers including all transducers of the plurality of transducers or a multitude thereof.

Different array designs are possible. For example, the transducers of the array can be activatable together or separately. Further, the array can be configured to run a first transducer or a first set of transducers of the array with a first set of operating parameters and a second transducer or a second set of transducers of the array with a second set of operating parameters.

The array designs can distinguish in the size and shape of the array.

The treatment device can include at least one array of a first design and at least one array of a second design.

The treatment device can include a single transducer in combination with at least one array.

Alternatively, the at least one transducer can be released from and attached to the carrier element.

“Released” means that the at least one transducer can be moved relative to the carrier element. This does not include necessarily that the at least one transducer becomes separate from the carrier element.

The at least one transducer can be attached, released and re-attached a plurality of times.

In particular, the at least one transducer can be attached at a position of the carrier element that is different from the position from which it was released.

In other words, a position of the at least one transducer on the carrier element can be changed. This means that the carrier element and the at least one transducer are configured in a manner that the at least one transducer can be arranged at different, ideally all, positions on the carrier element.

For example, the carrier element can include at least one fixation element to which the at least one transducer can be attached.

The fixation element(s) can be configured to allow for a change of the orientation of a transducer mounted to the carrier element via the fixation element.

The fixation elements can be configured to allow for the fixation of different kinds of transducer. Transducers can differ in their size, weight, and/or output signal generated, for example.

The fixation elements can be configured to allow for the fixation of the at least one transducer in choosable orientation.

For example, a surface of the carrier element, in particular a surface of the carrier element that is an outer surface when fastened on the body portion, can include the loops and the at least one transducer can include the hooks (and vice versa) of a hook-and-loop fastener.

For example, a surface of the carrier element, in particular a surface of the carrier element that is an outer surface when fastened on the body portion, can include pockets, in which the at least one transducer can be arranged, or it can include clamping means, for example an elastic cord, configured for clamping the at least one transducer to the carrier element.

In an embodiment the treatment device allows the determination of a position of the at least one transducer relative to the body portion when the carrier element is fastened on the body portion and the at least one transducer is mounted on the carrier element.

As mentioned above, embodiments of treatment devices according to the first aspect can include this feature in any embodiment, too.

Embodiments of treatment devices according to the first aspect can include any feature disclosed in relation to embodiments of the second aspect and vice versa.

For example, the treatment device can be configured to determine the position of the at least one transducer relative to the body portion when the treatment device is fastened to the body portion. For example, the carrier element can include conductor tracks that change their properties if a transducer is present at a track-specific position on or in the carrier element and/or the carrier element includes a fixation element configured to detect the presence of a transducer.

Alternatively or in addition to said configuration of the treatment device, the treatment device can allow for the determination of the position of the at least one transducer by equipping the transducer with an element that makes its position observable for an equipment for detecting said position. The equipment for detecting can be any device mentioned in relation to the equipment for determining the characteristic of the means for determining a geometric property.

For example, the at least one transducer can include a passive detectable element, such as a QR code, a labelling or a conspicuous colour. Alternatively or in addition, the at least one transducer can include an active detectable element, such as an emitter or a sensor, for example as disclosed with respect to the means for determining a geometric property of the body portion.

In principle, it can be sufficient to determine the position of the at least one transducer relative to the carrier element if the carrier element is used to determine a geometric property, in particular the shape, of the body portion.

Hence, the treatment device can include the means for determining the shape of the body portion to be stimulated and it can allow the determination of the position of the at least one transducer relative to the carrier element when the carrier element is fastened to the body portion. In this embodiment at least, the carrier element should not be moved between the determination of the shape of the body portion and the determination of the position of the at least one transducer. Otherwise, the determined position on the carrier element does not correspond to a position relative to the body portion that is concluded from the position on the carrier element.

As mentioned above, the at least one transducer can include an element that is detectable by a detector (sensor). The detector can be or comprised by the equipment for determining the characteristic of the means for determining a geometric property.

The determination of the position of the at least one transducer relative to the body portion (relative to the carrier element, as the case may be) can be determined by one of the approaches disclosed in relation to the determination of a geometric property of the body portion, namely a marker, sensor and/or emitter arranged on the at least one transducer and the related detection of the marker or an output of the sensor or emitter.

For example, the position of the marker and hence of the at least one transducer can be determined by taking at least one image including the at least one transducer when fastened on the body portion and a program configured to calculate the position of the at least one transducer.

The maker can be or include an identifier. This allows to determine which transducer is at which position.

The treatment device can include a controller.

The controller can be configured to control elements of the treatment device, such as the at least one transducer, the sensors, the emitters, the equipment for determining the characteristic of the means for determining a geometric property detector(s) etc.

As mentioned above, the treatment device receives the operating parameters and potentially further parameters of the set of treatment parameters from a remote computerized device. Hence, there is no need that the treatment device includes a computerized device configured to determine the set of treatment parameters or a portion thereof. Nevertheless, the controller can be configured to run a program, for example a program for at least one of determining the geometry of the body portion, determining the position of the at least one transducer, determining the type of the at least one transducer, validating a position of the at least one transducer, validating the type of the at least one transducer, validating a set of treatment parameters, validating a geometry of the body portion, adjusting a set of treatment parameters etc.

Another element of the treatment device can include the controller, for example the carrier element or the equipment for determining the characteristic of the means for determining a geometric property.

Alternatively, the controller can be a separate element of the treatment device that can establish a data connection to other elements of the treatment device, such as the at least one transducer, the sensors, the emitters, the equipment for determining the characteristic of the means for determining a geometric property and detector(s).

In an embodiment, the treatment device is a stand-alone device. This means it can carry out any steps needed to prepare and apply a stimulation of the body portion. However, this does not exclude that the treatment device can be connected to a remote device, for example for downloading updates and uploading information related to stimulations that have been carried out.

The treatment device can include communication means to transmit information from the treatment device to the remote computerized device and vice versa.

The computerized device can be any device owned by the user, such as its cell phone, tablet, PC etc.

The controller can include the communication means.

The computerized device can host and/or have access to the library, wherein the library includes at least one of data provided by other users of a treatment device and data provided by an operator of the library. The library can be the library disclosed with respect to any embodiment of the treatment device.

The computerized device can be configured to run a program, for example one of the programs listed above or a program for determining a set of treatment parameters or a portion thereof. In particular, it can be configured to run a program that needs a lot of resources, such as the determination of a set of treatment parameters.

In embodiments of the treatment device that are configured to determine a position of the at least one transducer relative to the body portion, and to transmit the position of the at least one transducer relative to the body portion to the computerized device, the position of the at least one transducer transmitted can become part of a set of treatment parameters that is transmitted to the treatment device after its determination, this means that is received (retrieved) by the treatment device.

In other words, the treatment device can be configured to receive (retrieve) a set of treatment parameters, in particular operational parameters for the at least one transducer, via the communication means, wherein the set of treatment parameters is determined in consideration of the determined position of the at least one transducer. The determined position can include information concerning the orientation of the transducer relative to the body portion and concerning potentially contact pressure.

In this embodiment, a method for determining a set of treatment parameters can be restricted to a determination of the operating parameters.

This embodiment often includes transducer(s) capable to stimulate the body portion by acoustic wave(s) that is/are attached to the carrier element in a fixed manner.

The treatment device can be capable to apply the set of treatment parameters, in particular to operate the at least one transducer with the operational parameters. In particular, the controller can be configured to operate the at least one transducer with the operational parameters

In an embodiment, the device is configured to receive a set of treatment parameters from the computerized device, wherein the set of treatment parameters includes a target position of the at least one transducer and wherein
The device includes at least two transducers, wherein the device is configured to activate the transducer that is closest to the target position during stimulation, wherein the other transducer remains in an off-state.
This feature is disclosed further below in relation to a variant of the embodiment including a device that is configured to receive a set of treatment parameters from a computerized device.
The carrier element includes position marks that help to arrange the at least one transducer at the target position.
This feature is disclosed further below in relation to a variant of the embodiment including a device that is configured to receive a set of treatment parameters from a computerized device.
The device is configured to determine that the at least one transducer is at the target position.

In a variant of the embodiment including a device that is configured to receive a set of treatment parameters from a computerized device, the treatment device includes the communication means to exchange data with the computerized device, wherein the treatment device is configured to transmit the geometric property of the body portion to the computerized device.

The geometric properties can be used by the computerized device to determine a set of treatment parameters that is subject specific, wherein in this embodiment a target position of the at least one transducer is determined together with other parameters of the set of treatment parameters, in particular together with the operational parameters.

The target position is the position that the at least one transducer should have with respect to the body portion so that an acoustic field needed for the given application is generated in the body portion when the other parameters of the set of parameters are applied to the treatment device, in particular when the operational parameters are applied to the at least one transducer.

In other words, the treatment device can be configured to receive (retrieve) a set of treatment parameters including or consisting of operational parameters for the at least one transducer and a target position of the at least one transducer via the communication means, wherein the set of treatment parameters is determined in consideration of the geometric property of the body portion.

Independent of the concrete embodiment, the treatment device can be capable to operate the at least one transducer with the operational parameters. In particular, the controller can be configured to operate the at least one transducer with the operational parameters.

Often, the geometric property is the shape of the body portion.

This embodiment often includes transducer(s) capable to stimulate the body portion by acoustic waves that is/are releasable and attachable to the carrier element, this means transducer(s) that can be rearranged on the carrier element.

The carrier element can include position marks that allow to arrange the at least one transducer at its target position.

The position marks can be passive, such as coordinate system, or it can be active, such as visual impression generated at the target position.

Alternatively, this embodiment can include a treatment device including a plurality of transducers and the treatment device can be configured to select the transducer(s) that is/are closest to the target position(s).

“Select” means in this case that only the selected transducer(s) are activated, wherein the non-selected transducer(s) remain or a set in an off-state. In other words, the operational parameters are applied to the selected transducer(s) only, wherein the non-selected transducer(s) are not operated during stimulation. If more than one transducer is selected, the selected transducers work together to generate the acoustic wave used for stimulation.

For example, an array can include the transducers and the acoustic wave generated by the array can be acoustic wave needed for the stimulation.

The device can include at least two transducers, wherein the device, in particular its controller, is configured to actuate the transducer that is closest to the target position during stimulation wherein the other transducer remains or is set in an off-state.

In an embodiment, the device is configured for validating a configuration of the device with respect to a set of treatment parameters by comparing at least one measured parameter of the treatment device with a related parameter of the set of treatment parameters.

A set of treatment parameters includes all parameters needed to configure and run the treatment device in a manner that an acoustic field is generated in the body portion, said acoustic field being able to stimulate the regions in the body portion that have to be stimulated in order to achieve an effect envisaged by the chosen application. Operational parameters of the at least one transducer, a target position of the at least one parameters and further parameters concerning the relative arrangement between the at least one transducer and the body portion, such as the orientation of the transducer and the contact pressure are examples of treatment parameters (see above).

For example, at least one of the following can be compared:
The measured position(s) of the transducer(s) with the target position(s) of the set of treatment parameters.
If such a comparison is applied, the determination of a position of the at least one transducer relative to the body portion can be considered as a part of a validation.
The selected transducer(s) with a related list of the set of treatment parameters.
The measured angle between the oscillation axis (axes) of the transducer(s) and the surface normal of the body portion at the position(s) of the transducer(s) with the target angle(s) of the set of treatment parameters.
The type of transducer with a list including types of transducers.

In embodiments, the validation is carried out immediately prior to the start of the stimulation.

The parameter to be measured for comparison with the related parameter of the set of treatment parameters determines which element of the treatment device is involved. In particular one of the following elements can be configured to validate a parameter of the set of treatment parameters: the controller, sensors, emitters, detectors, equipment for determining the characteristic of the means for determining a geometric property etc.

In an embodiment, the device includes an input element for receiving a user feedback related to an effect of the ultrasonic stimulation.

For example, the input element can be configured to allow a rating of the effect generated.

The input element can include a touch screen, a button etc., for example.

In addition or alternatively, the input element can be for receiving information related to an effect of the ultrasonic stimulation that is generated in an automated manner.

For this, the treatment device can include a sensor for supervising an effect of the ultrasonic stimulation. The sensor can be sensitive for a neuronal or electrical activity.

The treatment device can include an Electroencephalography (EEG) sensor.

The user feedback and/or the information generated in an automated manner can be used to adjust the set of treatment parameters.

In an embodiment, the device includes an input element for receiving (retrieving) information related to an effect of the ultrasonic stimulation, wherein the device includes communication means for transmitting the information to a library that is available for other users.

The information related to an effect of the acoustic wave stimulation can be the user feedback or the information generated in an automated manner, for example.

Further information can be transmitted together with the feedback to the library, such as the set of treatment parameters on which the stimulation is based, the measured parameters of the setup used and subject specific information, such as gender, age, weight, height, BMI, body fat content, fat percentage, muscle percentage etc., but also the geometric property of the body portion determined.

The measured position(s) of the transducer(s), the measured operational parameters, the measured orientation of the transducer(s) with respect to the body portion and the measured contact pressure are examples of measured parameters of the setup used.

The transmitted information can be used for improving continuously the set of treatment parameters transmitted to a treatment device for a specific application by improving continuously the data base for determining a set of treatment parameters.

The information related to an effect of the ultrasonic stimulation can be stored locally for being available for a future treatment of the same kind and/or of the same user.

The information can be stored in the treatment device, for example the controller, or an element owned by the user and connectable to the treatment device, such as the equipment for determining the characteristic of the means for determining a geometric property.

In an embodiment, the device includes at least one sensor for generating subject specific information other than the geometric property of the body portion.

At least some subject specific information can be generated by a user input. In other words, an input element can be configured to receive (retrieve) subject specific information from the user.

The input element for receiving subject specific information can be the same element as the input element for receiving a user feedback.

The input element receiving subject specific information or another input element can be configured for the selection of an application by the user.

In an embodiment, the geometric property includes the shape of a portion of the body portion, for example the shape of a bone, such as the skull or a long bone, or the arrangement of muscles, body fat etc.

In such an embodiment, the treatment device can include a transducer for exciting acoustic waves in the body portion and a transducer for detecting acoustic waves that are transmitted through and/or scattered in the body portion.

The transducer for exciting acoustic waves can be the transducer for detecting acoustic waves.

The transducer for exciting acoustic waves and the transducer for detecting acoustic waves can be different from the transducer capable to stimulate the body portion by acoustic waves in terms of acoustic waves generated and in terms of acoustic wave detection characteristics.

For example, the treatment device can include a second kind of transducer configured for applying acoustic waves to the body portion and to measure transmitted and/or scattered acoustic waves.

Alternatively, the treatment device can include a second kind of transducer configured for applying acoustic waves to the body portion and a third kind of transducer configured to measure transmitted and/or scattered acoustic waves.

The second and—as the case may be the third—kind of transducer can be different from the at least one transducer capable to stimulate the body portion by acoustic waves, the transducer being the first kind of transducer, in terms of acoustic waves generated and in terms of acoustic wave detection characteristics.

The second and—as the case may be third—kind of transducer can be arranged on a carrier element that is different from the carrier element on which the at least one transducer capable to stimulate the body portion by acoustic waves (i.e., the “first kind of transducer”) is arranged.

However, the treatment device can include both, the carrier element for the first kind of transducer and the carrier element for the second and—as the case may be—third kind of transducer.

In embodiments including a carrier element that is formed according to a portion of the body portion to be treated at least, for example an accordingly formed rigid material, the carrier element can include a fixation element per transducer needed for a specific application, wherein each fixation element is arranged on the carrier element at a target position for the specific application.

For example, the fixation element(s) can be integral with or firmly mounted to the carrier element that is formed according to a portion of the body portion, wherein the position of the fixation element(s) on the carrier element corresponds to the target position(s) of the transducer(s) for a selected application (treatment) and for a carrier element that is fastened on the body portion in a manner that the carrier element that is formed according to a portion of the body is adjacent to said portion of the body.

Alternatively, the carrier element includes an arrangement of a plurality of fixation elements that are integral with or firmly mounted to the carrier element, for example to the portion that is formed according to a portion of the body portion, and that allows by their number and arrangement the fixation of the at least one transducer at target positions of various applications (treatments).

The plurality of fixation elements can be optimized for a limited number of predefined applications.

Independent of the concrete embodiment of the treatment device including at least one fixation element, the device may include means for detecting the correct fixation of the at least one transducer in the at least one fixation element. In other words, the at least one fixation element and/or the at least one transducer can be configured to ensure a predefined position and a predefined orientation of the at least one transducer.

Independent of the concrete embodiment, the carrier element, in particular the rigid portion thereof (if present), can be manufactured by a rapid manufacturing process, such as milling, turning, casting or an additive manufacturing process such as printing, for example 3D-printing.

The invention concerns further a system including a carrier element, at least one transducer, communication means (a communication unit) and a computer program including instructions which, when executed by the remote computerized device, in particular the computerized device owned by the user, cause the computerized device to exchange data with the communication means.

The computer program can include any further instructions which, when executed by the remote computerized device, cause the computerized device to fulfil any function disclosed with respect to the treatment device and/or the method for setting-up, such as the detection of the shape of the body portion to be treated and/or of the position of the at least one transducer by using the camera of the computerized device, exchanging data with a further remote computerized device, controlling the treatment device, providing a user interface etc.

In an embodiment, the remote computerized device is not part of the system.

The system may include the treatment device in any embodiment disclosed, wherein the carrier element, the at least one transducer and the communication means are the carrier element, the at least one transducer and the communication means of the treatment device.

The computer program (also called “application” or “app”) is loadable into an internal memory of the computerized device and includes computer-executable instructions to cause one or more processors of the computerized device to control the computerized device in a manner that it is able to fulfil any function disclosed with respect to the treatment device and/or the method for setting-up.

The computer program can be stored on a computer readable medium having the computer-executable instructions recorded thereon. The computer readable medium preferably is non-transitory, that is, tangible.

In particular in embodiments having a carrier element with a shape that is specific to a body portion to be treated, for example by including rigid material that is formed according to a portion of the body portion to be treated, and that include at least one fixation element being integral with or firmly mounted to the carrier element, at least one object of the invention can be met by the carrier element.

Hence, the invention also relates to a carrier element that is formed according to a portion of the body portion to be treated at least, wherein
the carrier element includes a fixation element for at least one transducer, wherein the number of fixation element(s) and the arrangement of the fixation element(s) are adapted to a specific acoustic wave stimulation; or
the carrier element includes an arrangement of a plurality of fixation elements that allows fixation of at least one transducer at target positions of various acoustic wave stimulations.

In an embodiment, the carrier element is formed according to a least a portion of the body portion of an individual subject, said body portion being the body portion to be treated. In other words, the carrier element is not only formed to fit at least a portion of a leg, an arm, a neck, a head, a back etc. but to fit at least a portion of a leg, an arm, a neck, a head, a back etc of a given patient.

In an embodiment, the carrier element includes rigid material and it is the rigid material that is formed according to the portion of the body portion to be treated. Said body portion may be the body portion of a given patient.

The carrier element can be a part of a kit of parts that further includes transducers in the number and of the type used for a stimulation (application) selected by a user or for a plurality of stimulations (application) that can be carried out on the body portion to which the carrier element correspond.

The invention also relates to a method of manufacturing a treatment device, in particular a portable treatment device. The method includes the steps of:
Determining at least a portion of the shape of the body portion to be treated;
Selecting a treatment of the body portion;
Determining a target position of at least one transducer relative to the body portion, wherein the number of transducers and target positions as well as the target positions depend on the selected treatment and the shape determined;
Providing a carrier element and transducers in said number, wherein the carrier element is equipped to arrange the transducers at the target positions.

In an embodiment, the step of determining at least a portion of the shape of the body portion, the step of selecting a treatment of the body portion and the step of determining a target position is carried out by a practitioner.

In an embodiment, the method includes further a step of arranging the at least one transducer at the at least one target position. This step can be carried out by the patient (end-user), by the manufacturer or by the practitioner.

If the step of arranging is carried out by the manufacturer, the treatment device provided by the manufacturer may be a fully assembled device.

In an embodiment, the carrier element is formed at least according to the portion of the shape of the body portion to be treated.

For example, the carrier element can be formed at least according to the portion of the shape of the body portion of an individual subject to be treated.

In an embodiment of the method of manufacturing the carrier element provided is formed according to a (individualized or non-individualized) portion of the body portion to be treated and includes at least one fixation element integral with or firmly mounted to the carrier element. For example, the carrier element can include rigid material that is formed according to a (individualized or non-individualized) portion of the body portion to be treated and with which the fixation element is integral or to which the fixation element is mounted.

For example, the step of determining the shape of the body portion to be treated includes taking a picture of the body portion or making a scan of it and the step of providing a carrier element includes manufacture of the carrier element using the determined shape of the body portion and rapid manufacturing process, such as milling, turning, casting or an additive manufacturing process such as printing, for example 3D-printing.

As mentioned, methods for determining, validating and/or adjusting the set of treatment parameters for acoustic wave stimulation are discussed in detail in the application entitled “Treatment parameters for acoustic wave stimulation” filed by the same applicant at the same date as the present application. However, such methods can include a step of generating a digital 3D model including a model of the surface of the body portion to be stimulated. If this is the case, the digital 3D model can be used to manufacture the carrier element.

The invention further relates to methods for setting up a device for acoustic wave stimulation of a body portion. A method for operating a device for acoustic wave stimulation of a body portion can include the method for setting up in any embodiment.

The methods are in particular, but not exclusively, suitable for setting up a treatment device according to any embodiment as described above.

Concrete embodiments of the method for setting up the treatment device can vary significantly. For example:
In an embodiment, the treatment device can be in a configuration in which an improper use may have severe consequences. Often, this is the case if the treatment device is a treatment device for medical indications. In this embodiment, a practitioner is usually involved directly in setting up the treatment device.
An embodiment for setting up a treatment device of this kind may include a step of receiving a carrier element and at least one transducer from a manufacturer and a step of arranging the at least one transducer on the carrier element, wherein the step of arranging the at least one transducer on the carrier element is carried out by the practitioner.
Optionally, the practitioner can provide reference points (landmarks) for ensuring a correct arrangement of the treatment device on the body portion to be treated. Optionally, the practitioner can determine the operating parameters. The practitioner can provide the operating parameters in a manner that it can be received by the treatment device later. In other words, the operating parameters are subject specific. In cases in which the treatment device is for one medical indication only, the treatment device may be received fully assembled. In this case, there may be no step of arrange the at least one transducer relative to the carrier element.
In an embodiment, the treatment device can be in a configuration in which an improper use may affect the efficacy of the treatment but has no severe consequences for the user. Often, this is the case if the treatment device is a treatment device for well-being. In this embodiment, there is usually no need for a practitioner being involved directly in setting up the treatment device.
An embodiment for setting up a treatment device of this kind may include a step of receiving a carrier element and at least one transducer from a manufacturer and a step of arranging the at least one transducer on the carrier element, wherein the step of arranging the at least one transducer on the carrier element is carried out by a non-practitioner, such as the end-user or a person that offers a treatment of this kind to an end-user.
The step of arranging can include receiving information concerning the arrangement of the at least one transducer on the carrier element. In particular, it can include any step disclosed with respect to the embodiment in which a set of treatment parameters is received. Alternatively or in addition, it can include receiving information concerning the arrangement of the at least one transducer on the carrier element from a practitioner.
Optionally, a correct arrangement of the treatment device on the body portion to be treated can be ensured by reference points (landmarks) provided by a practitioner and/or received from a library.
A correct arrangement of the at least one transducer and/or treatment device may be ensured by including any step disclosed with respect to the determination of a geometric property of the body portion to be treated and/or the determination of a position of the at least one transducer.
Optionally, the operating parameters may be provided by a practitioner in a manner that it can be received by the treatment device later.
In embodiments, in which the carrier element was manufactured based on data generated by a practitioner, said practitioner can be the practitioner providing the operating parameters.
However, the operating parameters may be determined by a computer-implemented method configured to select the operating parameters from a plurality of operating parameters that are stored in a library
In cases in which the treatment device is for one medical indication only, the treatment device may be received fully assembled. In this case, there may be no step of arrange the at least one transducer relative to the carrier element.

Independent of the concrete embodiment of the method for setting up the treatment device, a method for setting up a device for acoustic wave stimulation of a body portion, wherein the device includes at least one transducer, a carrier element and communication means, includes the step of receiving operating parameters for the at least one transducer from a remote computerized device.

The method can include a step of providing the treatment device, in particular a treatment device according to any embodiment disclosed.

The remote computerized device is the remote computerized device according to any embodiment described above.

The operating parameters received may have been determined by a practitioner or by a computer-implemented method configured to select the operating parameters from a plurality of operating parameters that are stored in a library, for example. Whether the operating parameters received are determined by a practitioner or by a computer-implemented method may depend on the application. If the application concerns a medical indication, the operating parameters have preferably been determined or at least approved by a practitioner. If the application concerns well-being, for example, the operating parameters have preferably been determined by the computer-implemented method.

The computer implemented method may be realized as a computer program including instructions which, when executed by a computer, cause the computer to select the operating parameters from a plurality of operating parameters that are stored in a library.

The computer-implemented method may be the method disclosed in the application entitled “Treatment parameters for acoustic wave stimulation” (CH application number 00394/19) filed by the same applicant at the same date as the present application.

In embodiments, in which the operating parameters received are part of a set of treatment parameters

In embodiments, the operating parameters received are part of a set of treatment parameters in the sense discussed above. In other words, the data received from the remote computerized device can include information concerning the arrangement of the at least one transducer besides the operating parameters (pulse duration, pulse length, pulse repetition rate, intensity etc.).

The operating parameters (the set of treatment parameters as the case may be) received are the operating parameters (set of treatment parameters) determined by the computerized device, as described above.

Embodiments in which the operating parameters received are part of a set of treatment parameters can further include at least one of:
The set of treatment parameters received includes a target position of the at least one transducer relative to the body portion, and the method includes a step of arranging the at least one transducer on the carrier element and at the target position.
In other words, the position(s) of the transducer(s) relative to the body portion can be a variable during determination of the set of treatment parameters.
A treatment device that is operated according to this embodiment of the method needs transducer(s) that can be released and (re-) attached to the carrier element.
The correct arrangement of the at least one transducer can be supported by the treatment device, in particular the carrier element can include marks as disclosed with respect to the treatment device, for example.
The treatment device, in particular the carrier element and optionally the at least one transducer, can include a fixation element that allow for a release and (re-) attachment of the at least one transducer at various positions, as disclosed with respect to the treatment device.
The step of determining a position of the at least one transducer (2) relative to the body portion can be an important validation step of embodiments including the step of receiving a target position and the step of arranging the at least one transducer at the target position.
The device includes a plurality of transducers, wherein the set of treatment parameters received includes at least one target position of a transducer, and wherein the method includes the step of activating the transducer that is closest to a target position, wherein any transducer that is not closest to a target position remains in an off-state. In other words, the treatment device includes a plurality of transducers distributed over the carrier element and the controller is configured to select the transducer(s) that are closest to the target position(s) of the received set of treatment parameters. Optionally, this embodiment of the method can include a step of rearranging the at least one transducer, nevertheless. However, the range of rearrangement of the at least one transducer can be limited. For example, it can be a fine-tuning of at least one of the position, of the orientation and of the contact pressure of the at least one transducer.
The at least one transducer can be fixed to the carrier element in a manner that allows for the fine-tuning. Optionally, the fixation of the transducer can prevent a complete separation of carrier element and treatment device.
A step of validating a configuration of the treatment device with respect to the set of treatment parameters received by comparing a measured parameter of the treatment device with a related parameter of the set of treatment parameters.
For example, the position of the at least one transducer and/or its orientation relative to the body portion is validated. In this case, the step of determining a position of the at least one transducer relative to the body portion can be a substep of the validation step.
The step of validating can include the use of the treatment device in any embodiment configured for validation of a configuration of the device.
Optionally, the step of validating can trigger a step of adjusting the set of treatment parameters if an outcome of the step of validating is negative.
The outcome of the step of validating can be negative if a measured parameter distinguishes from the related parameter in the set of treatment parameters by more than a pre-set value.
The step of validating can include a substep of determining a difference between a measured parameter and the related parameter in the set of treatment parameters and the sub step of comparing the difference with a pre-set value.
The step of validating and the step of adjusting—as the case may be—can be carried out before a step of applying the set of treatment parameters to the treatment device or during the step of applying the set of treatment parameters to the treatment device, depending on the parameter that is validated.

As pointed out with respect to the treatment device, there are different aspects that may help to reduce the parameter space that a user of the treatment device has to handle. Consequently, embodiments of the method can differ in dependence of the concrete realization of the treatment device.

Embodiments of the method that are in particular suitable for setting-up treatment devices according to or including the first aspect can have the following steps in common:
Fastening the carrier element to the body portion to be stimulated.
Determining a geometric property of the body portion.
The geometric property can be determined in any manner disclosed with respect to the treatment device.
In particular, the means for determining a geometric property of the body portion are used in the step of determining a geometric property of the body portion.
In embodiments, it is the shape of the body portion that is determined, at least.
Transmitting the geometric property to a computerized device capable to determine operating parameters (a set of treatment parameters as the case may be) for the treatment device.
The operating parameters (set of treatment parameters) is the set of treatment parameters discussed above.
The computerized device can be one of the remote device and the device owned by the user, such as its cell phone, tablet, PC etc. as disclosed above, for example.
The computerized device can be configured to determine operating parameters (a set of treatment parameters as the case may be) for the application given by the body portion and the selection of the user.
The computerized device considers the geometric property transmitted when determining the operating parameters (set of treatment parameters).
Information concerning the desired treatment, the body portion to be treated or subject specific data can be transmitted to the computerized device together with the geometric property.

Embodiments of the method that are in particular suitable for setting-up treatment devices according to or including the second aspect can have the following steps in common:
fastening the carrier element to the body portion to be stimulated; and
determining a position of the at least one transducer relative to the body portion when the at least one transducer is fastened to the carrier element and the carrier element is fastened on the body portion.
a step of transmitting the position of the at least one transducer to the computerized device.
The computerized device can be one of the remote device and the device owned by the user, such as its cell phone, tablet, PC etc. as disclosed above, for example.
The computerized device can be configured to determine operating parameters (a set of treatment parameters as the case may be) for the application given by the body portion and the selection of the user.
The computerized device considers the position of the at least one transducer transmitted when determining the operating parameters (set of treatment parameters).

In embodiments, the method according to the second aspect includes at least one of:
The step of determining a position of the at least one transducer relative to the body portion can include any substep related to the detection of an active or passive element as disclosed in relation to treatment device being configured to determine a position of the at least one transducer relative to the body portion.
In particular, the step of determining a position can include a substep of taking an image. This also includes a substep of taking a sequence of images, such as to perform a scan.
Alternatively or in addition, the step of determining a position can include a sub step of determining the presence of the at least one transducer in a fixation element of the carrier element.
The operating parameters received are part of a set of treatment parameters received that includes the position of the at least one transducer as target position. In other words, the determined position of the at least one transducer is considered to be fix and a set of treatment parameters is calculated on the assumption of a fix transducer (of fixed transducers, as the case may be). The at least one transducer can be fastened to the body portion by the carrier element, for example in a manner as disclosed with respect to the treatment device.

Embodiments in line with the first and second aspects can be part of a method for operating the treatment device. In particular, the steps in common with the first aspect and/or the steps in common with the second aspect can be characteristic for a method for operating a treatment device for acoustic wave stimulation, wherein the device includes at least one transducer and a carrier element.

Embodiments of the method, in particular embodiments according to the second aspect, can be or can be a part of a validation step of a more general method for setting up and/or of a method for operating a treatment device, in many cases. In such cases, the method can be considered as a method for validating a set of treatment parameters or a portion thereof.

An embodiment of the method according to the second aspect can include the three characteristic steps of the methods according to the first aspect listed above. In addition or alternatively, it can include any further step disclosed in relation to embodiments according to the first aspect, unless otherwise specified.

Similarly, an embodiment of method according to the first aspect can include the step of determining a position of the at least one transducer relative to the body portion of the second aspect listed above. In addition or alternatively, it can include any further step disclosed in relation to embodiments according to the second aspect, unless otherwise specified

In an embodiment, the operating parameters (set of treatment parameters as the case may be) is applied to the treatment device to generate the acoustic waves needed to affect a stimulation that is in agreement with the desired application. However, a step of applying the operating parameters (set of treatment parameters) can be carried out subsequent to at least one of a step of validating the operating parameters (set of treatment parameters), a step of validating the position of the at least one transducer, and a step of adjusting the operating parameters (set of treatment parameters). Alternatively or in addition, the step of applying the operating parameters (set of treatment parameters) can include the substep of adapting the operating parameters (set of treatment parameters). This means that the operating parameters (set of treatment parameters) is adjusted during the step of applying the operating parameters (set of treatment parameters). The step of applying can be interrupted for an adjustment of the operating parameters (set of treatment parameters).

In embodiments, the method can include at least one of the following steps in addition to any other step disclosed above or in addition to any combination of steps disclosed above:
A step of receiving a user feedback related to an effect of the ultrasonic stimulation.
The step of receiving a user feedback can include the use of the treatment device in any embodiment including an input element for receiving a user feedback.
The step of receiving a user feedback can trigger a step of adjusting the set of treatment parameters if the user feedback rates the effect of the ultrasonic stimulation as insufficient.
A step of receiving at least one of a start signal and a stop signal from the computerized device. The start signal enables the device to carry out the treatment whereas the stop signal prevents a start of the treatment.

The steps of the method in any embodiment can be carried out by the treatment device, in particular by an element of the treatment device configured to carry out a specific step of the method, if not otherwise stated.

Accordingly, the treatment device can be configured to carry out any step of the method disclosed.

Further optional steps of the method arise out of the description and the claims of the treatment device.

For example, if the treatment device (or an element thereof) is configured to carry out an action and/or capable to carry out an action, the method can include a step of carrying out said action.

### BRIEF DESCRIPTION OF THE DRAWINGS

The subject matter of the invention will be explained in more detail in the following text with reference to exemplary embodiments that are illustrated in the attached drawings, which schematically show:

FIG. 1 an exemplary embodiment of a treatment device;

FIG. 2 an exemplary embodiment of a carrier element of a treatment device;

FIG. 3 a further exemplary embodiment of a treatment device;

FIGS. 4-5 further exemplary embodiments of carrier elements

FIG. 6 a flow chart of a method for setting up a treatment device for acoustic wave stimulation

FIG. 7 a flow chart of an exemplary embodiment of the method for setting up a treatment device for acoustic wave stimulation of a body portion;

FIG. 8 a flow chart of a method for setting up a treatment device for acoustic wave stimulation of a body portion including steps related to the first aspect for reducing the parameter space to be handled by a user;

FIG. 9 a flow chart of a method for setting up a treatment device for acoustic wave stimulation of a body portion including steps related to the second aspect for reducing the parameter space to be handled by a user;

FIGS. 10-14 flow charts of exemplary embodiments of methods for setting up a treatment device for acoustic wave stimulation of a body portion; and

FIG. 15 a flow chart of an exemplary embodiment of a method for manufacturing a treatment device.

### DETAILED DESCRIPTION OF THE INVENTION

FIG. 1 shows an exemplary embodiment of a treatment device. The device shown includes a carrier element 1 essentially made from a flexible material. The carrier element 1 includes further a fastening element 9 arranged and equipped for fastening the carrier element on a body portion to be stimulated.

In the embodiment shown, two transducer 2 configured to generate an acoustic wave are mounted on the carrier element 1. The transducers include a detectable element 4, namely a QR code, on a surface of the transducers 2 that is visible when the carrier element 2 is fastened on the body portion.

The carrier element 1 includes means for determining a geometric property of the body portion when the carrier element 1 is fastened to the body portion. In the embodiment shown, the means are given by a grid arranged on a surface of the carrier element 1 that is visible when the carrier element 2 is fastened on the body portion.

The treatment device shown includes a controller 5 and communication means 6, namely wireless communication means.

The communication means 6 are configured to exchange data with a remote computerized device 11. In the embodiment shown, the remote computerized device 11 is computerized device owned by the user, for example a cell phone (as shown), a tablet, a personal computer, etc.

The cell phone is able to communicate with the communication means 6.

In the embodiment shown, an application (“app”) is provided together with the treatment device. This app is configured in a manner that components of the remote computerized device can be used, in particular during setting-up and operation of the treatment device. This has the advantage that the treatment device itself does not need such components and that there is no need to provide such components together with the treatment device.

In the embodiment shown, an equipment 18 for determining the characteristic of the means 3 for determining a geometric property of the body portion is not provided by or together with the treatment device but said equipment is the camera of cell phone.

The computerized device 11 is able to detect and interpreted the detectable element 4 via its camera 18, in the embodiment shown. In particular, the computerized device 11 is able to do this thanks to the app installed on it.

Further, a user interface 12 configured to control the treatment device by the user and including an input element for receiving a user feedback is not provided by or together with the treatment device but the user interface 12 is provided by the cell phone.

In the embodiment shown, the cell phone is the remote computerized device 11 providing a set of treatment parameters via the app installed on the cell phone and/or via communication with a further remote computerized device 7.

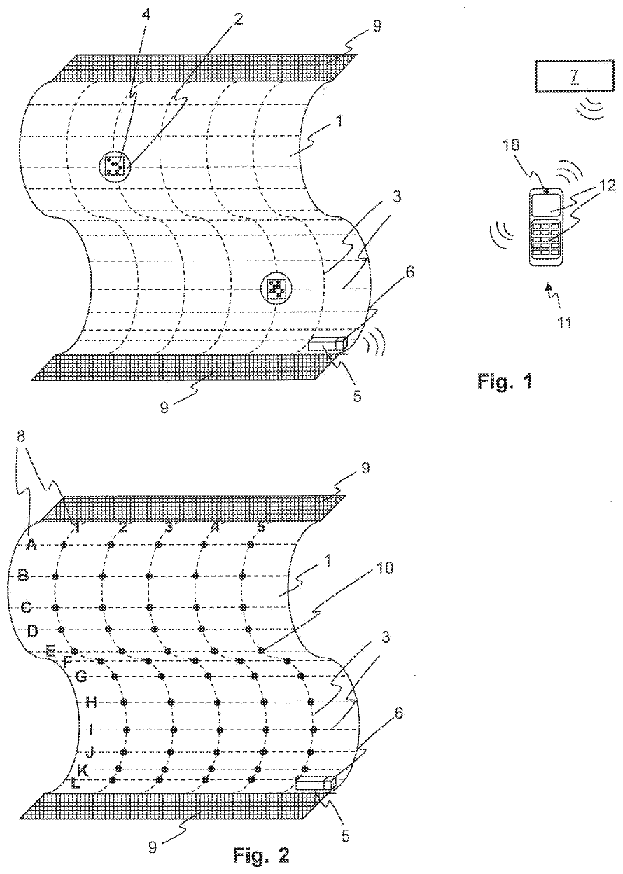

FIG. 2 shows an exemplary embodiment of the carrier element 1 that differs from the carrier element 1 shown in FIG. 1 by including a plurality of firmly mounted fixation elements 10 and by including marks 8 that enable a user to arrange and fix a number of transducers indicated by a set of treatment parameters at positions on the carrier element 1 indicated by the set of treatment parameters.

The portions of the set of treatment parameters that are relevant for the user to setup the treatment device, in particular number and position of the transducers, can be displayed to the user in the user interface 12.

In an alternative embodiment not explicitly shown in FIG. 2, transducers 2 are firmly mounted in the fixation elements 10 and the controller 5 is able to activate the transducers 2 as indicated by the set of treatment parameters.

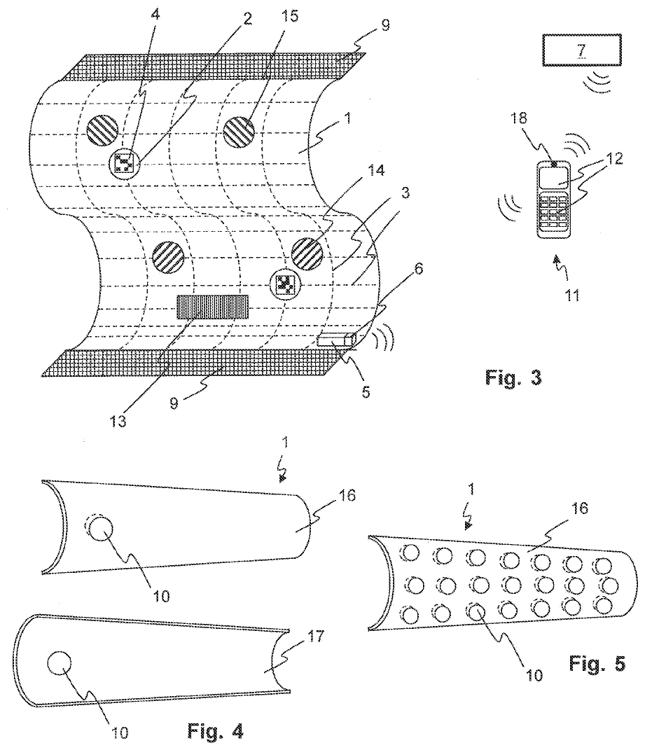

FIG. 3 shows an exemplary embodiment of the treatment device that differs from the treatment device shown in FIG. 1 by including means for determining further properties of the body portion to be stimulated.

In the embodiment shown, these further means include one or a plurality of transducers 14 for exciting acoustic waves in the body portion and one or a plurality of transducers 15 for detecting reflected and/or scattered acoustic waves.

The acoustic waves generated by the transducer(s) 14 for exciting acoustic waves in the body portion have characteristics that allow for the determination of acoustic properties within the body portion, such as the propagation speed, damping, the presence of scattering centers, or the position of changes in acoustic properties.

The controller 5 or any computerized device of the treatment device or in communication to it, can be configured to analyse the signals observed by the transducers 15 for detecting reflected and/or scattered acoustic waves.

In the embodiment shown, the further means for determining further properties of the body portion to be stimulated include a sensor 13 for recording subject specific information other than the geometric or acoustic properties of the body portion. Blood flow, neural activity and pulse are examples of such subject specific information.

The controller 5 or any computerized device of the treatment device or in communication to it, can be configured to analyse the subject specific information recorded. In particular, the subject specific information can be suitable for observing an effect generated by the stimulation. Hence, the subject specific information can be suitable for use in a feedback loop (control loop) for adjusting treatment parameters.

FIG. 4 shows two different views of an exemplary embodiment of the carrier element 1 including a portion 16 of rigid material that forms the negative of the body portion to be stimulated, this means that it includes a surface 17 that forms the negative of the surface of the body portion to be stimulated.

A carrier element 1 is shown in a simplified manner that fits to the forearm of a specific user.

The carrier element 1 of FIG. 4 is not only specific to a subject, this means it does not only include a surface 17 that forms a nearly perfect negative of the forearm of that subject (but generally not of another subject), but it is also specific to a treatment. This means, the fixation element 10 and the transducer 2 (when mounted to the carrier element 1) have a position that corresponds to a target position of a specific treatment after mounting the carrier element 1 on the body portion.

FIG. 5 shows a carrier element 1 that differs from the carrier element shown in FIG. 4 by including a plurality of fixation elements 10. This means that the carrier element 1 of FIG. 5 is specific to a user but not specific to a treatment. Rather, the transducers can be mounted at positions and in a number that correspond to the set of treatment parameters for one of various possible treatments.

Mounting of the transducers can be supported by markers 8 or the transducers 2 can be firmly mounted to the fixation elements 10 and the controller 5 can be configured to activate the transducers 2 as indicated by the set of treatment parameters.

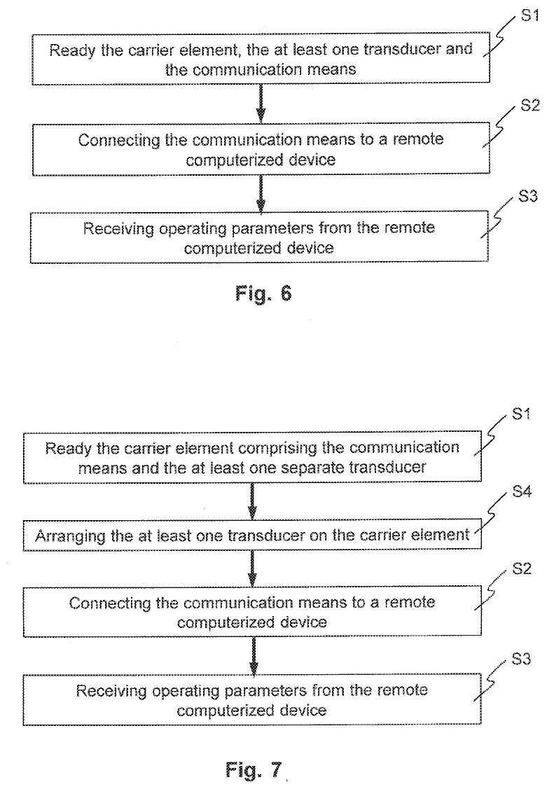

FIG. 6 shows a flow chart of basic steps of a method for setting up a treatment device. The basic steps are:
A step S1 of ready the carrier element 1, the at least one transducer 2 and the communication means 6.
The manner how the carrier element 1, the at least one transducer 2 and the communication means 5 are readied may depend on the manner these components are provided.
For example, the components may be provided in a fully assembled treatment device. In this case, the step S2 of ready the carrier element 1, the at least one transducer 2 and the communication means 6 may be a step of ready the treatment device. The treatment device provided can be the treatment device according to any embodiment disclosed.
For example, the components may be provided separately or some of said components, in particular the at least one transducer, may be provided separately from the other components. In this case, the step S2 of ready the carrier element 1, the at least one transducer 2 and the communication means 6 may be a step of ready said components in the state provided by a manufacturer.
For example, the at least one transducer 2 and the communication means 6 are provided by the manufacturer and the carrier element 1 has to be manufactured elsewhere, for example by the end-user, a practitioner or a medical facility. In this case, the step S2 of ready the carrier element 1, the at least one transducer 2 and the communication means 6 may be a step of ready the at least one transducer 2 and the communication means 6 provided by the manufacturer and of manufacturing the carrier element 1.
A step S2 of connecting the communication means 6 to a remote computerize device, such as the computerized device 11 owned by the user or another remote computerized device 7, for example a remote computerized device on which a library including a plurality of set of treatment parameters or a portion thereof is stored.
A step S3 of receiving operating parameters from the remote computerized device.

FIG. 7 shows a flow chart of an exemplary embodiment of the method for setting up a treatment device according to any embodiment disclosed. In the embodiment shown, a number of transducers 2 that corresponds to the number of transducers needed for a given treatment or a plurality of given treatments is provided separately from the carrier element 1 and the communication means 6 by the manufacturer. In other words, the communication means 6, and usually other components, such as the controller 5 and the fixation elements 10 and optionally at least one of the means 3 for determining a geometric property, the sensor 13 for subject specific information, the transducer(s) 14 for exciting, the transducer(s) 15 for detecting etc., are integral with or mounted to the carrier element whereas the transducer(s) 2 are separate from the carrier element 1.

In this embodiment, the step S1 of ready the carrier element 1, the at least one transducer 2 and the communication means 6 corresponds to a step of ready the transducer(s) 2 and the carrier element including the pre-assembled components of the later treatment device.

With respect to the steps disclosed in the embodiment of FIG. 6, the embodiment shown in FIG. 7 includes a further step S4 of arranging the transducer(s) 2 on the carrier element 1.

The step S4 of arranging includes a sub step of positioning and aligning the transducer(s) 2 on the carrier element 1 and a sub step of fastening the transducer(s) with the alignment at the position to the carrier element 1.

The concrete realization of the step S4 of arranging can depend on the envisaged treatment (application) or envisaged treatments (applications).

For example, if the treatment(s) are of a kind that an incorrect arrangement of the transducer(s) may have severe consequences for the user, the step S4 of arranging the transducer(s) may be carried out by a practitioner. In particular in this case, the method may further include a step of providing landmarks for correct positioning of the carrier element 1 on the body portion to be treated.

For example, if the treatment(s) are of a kind that an incorrect arrangement of the transducer(s) does not have severe consequences for the user, the step S4 of arranging the transducer(s) may be carried out by end-user. In particular in this case, the method may include receiving information concerning the position(s) and optionally the alignment(s) of the transducer(s). This information may be provided by a practitioner via the remote computerized device or it may be provided by the app, for example.

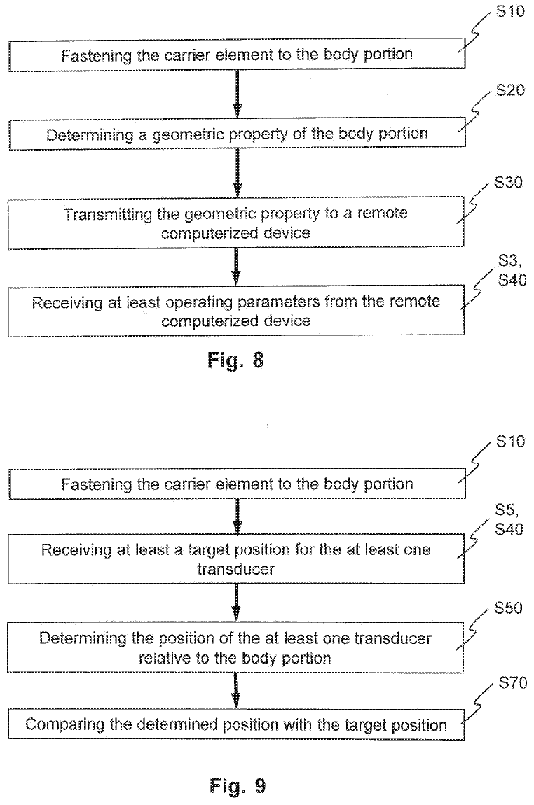

FIG. 8 shows a flow chart of an embodiment of the method for setting up a treatment device, said embodiment including steps related to the first aspect for reducing the parameter space to be handled by a user. The embodiment comprises:
A step S10 of fastening the carrier element 1 to the body portion to be stimulated.
A step S20 of determining a geometric property of the body portion.
A step S30 of transmitting the geometric property to the remote computerized device (7, 11), wherein the remote computerized device is capable to determine the operating parameter or—as the case may be—a set of treatment parameters for the treatment device.
The step S3 of receiving the operating parameters or a step S40 of receiving the set of treatment parameters form the computerized device, wherein the set of treatment parameters includes the operating parameters.

As a rule, the embodiment shown in FIG. 8 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIG. 7.

As pointed out above, there are embodiments of the treatment device, in particular of the carrier element 1, that do not need the step S20 of determining a geometric property of the body portion because fastening the treatment device on the body portion does not influence the body portion and because the geometric properties needed are determined without using the treatment device.

FIG. 9 shows a flow chart of an embodiment of the method for setting up a treatment device, said embodiment including steps related to the second aspect for reducing the parameter space to be handled by a user. The embodiment comprises:
The step S10 of fastening the carrier element 1 to the body portion to be stimulated.
A step S5 of receiving a target position for the at least one transducer 2 or the step S40 of receiving a set of treatment parameters form the remote computerized device (7, 11), wherein the set of treatment parameters includes the target position. In particular, a target position may be received for each transducer needed for a selected treatment during the step S5 of receiving a target position or the step S40 or receiving a set of treatment parameters.
A step S50 of determining the position of the needed transducer(s) 2 relative to the body portion.
Generally, this step S50 is carried out when the needed transducer(s) 2 are fastened to the carrier element 1 and the carrier element 1 is fastened on the body portion.
A step S70 of comparing the determined position(s) with the target position(s).
Any computerized device of the treatment device, for example the controller 5, or in connection to the treatment device can be configured to carry out the step S70 of comparing.
In dependence of the computerized device configured to carry out the step S70 of comparing, the method can include a step of transmitting the position(s) of the transducer(s) to the computerized device.

Optionally, the method according to FIG. 9 can include the further step of generating a stop signal preventing the treatment device from carrying out a treatment or the further step of generating a start signal enabling the treatment device to start a treatment, in particular a treatment based on the set of treatment parameters received. The difference between the determined position(s) and the target position(s) of the set of treatment parameters can be a criterion for deciding if a stop signal or a start signal is generated.

Optionally, the computerized device (5, 7,11) generates an adjusted set of treatment parameters if the difference between the determined position(s) and the target position(s) of the set of treatment parameters is larger than a predefined value. In this case, the method can include a second step S5 of receiving a target position for the at least one transducer 2 or a second step S40 of receiving a set of treatment parameters from the remote computerized device (7, 11). Again, a target position may be received for each transducer needed for a selected treatment during the step S5 of receiving a target position or the step S40 or receiving a set of treatment parameters

As a rule, the embodiment shown in FIG. 9 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIGS. 7 and 8 (if not present already).

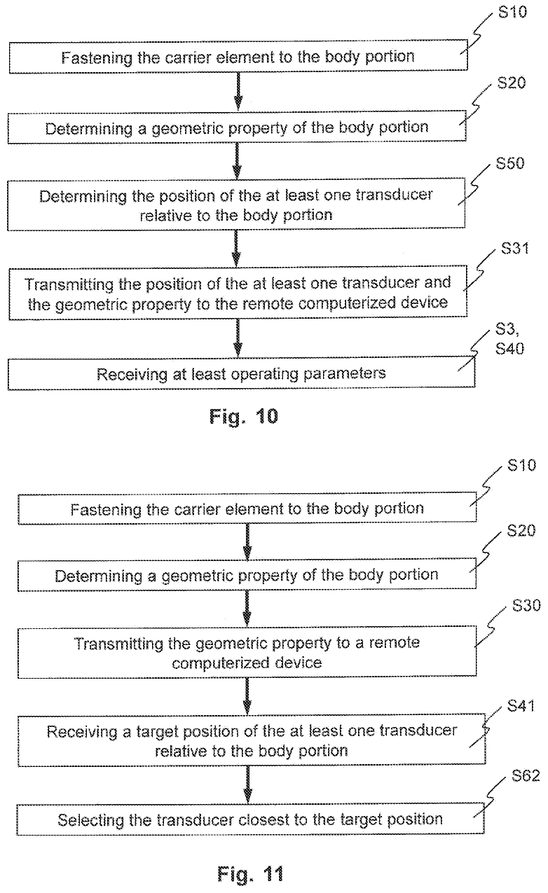

FIG. 10 shows an exemplary embodiment of the method for setting up the treatment device, wherein the embodiment shown differs from the embodiment shown in FIG. 8 by the additional step S50 of determining the position(s) of the needed transducer(s) 2 relative to the body portion and by transmitting not only the geometric property determined but also the position(s) determined in a step S31 of transmitting.

The embodiment of FIG. 10 allows the determination of operating parameters or a set of treatment parameters by the computerized device (7,11) that considers both the geometric property of the body portion to be stimulated and the position(s) of the available transducer(s) after fastening the carrier element 1 to the body portion.

The method of FIG. 10 can be used for determining a first set of operating parameters or a first set of treatment parameters for the selected application or it can be used for adjusting the operating parameter or a set of treatment parameters.

As a rule, the embodiment shown in FIG. 10 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIGS. 7-9 (if not present already).

The method of FIG. 11 is in particular suitable for treatment devices that include a plurality of transducers 2 that are firmly mounted to the carrier element 1. Hence, the exemplary embodiment of FIG. 11 includes a step S62 of selecting the transducer(s) that is/are closest to target position(s) received in a related step S41 of receiving. The target position(s) received are often part of a set of treatment parameters received.

In dependence of the realization of the treatment device, in particular of the carrier element 1, the step S20 of determining a geometric property and the step S30 of transmitting the geometric property can be omitted.

As a rule, the embodiment shown in FIG. 11 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIGS. 7-10 (if not present already).

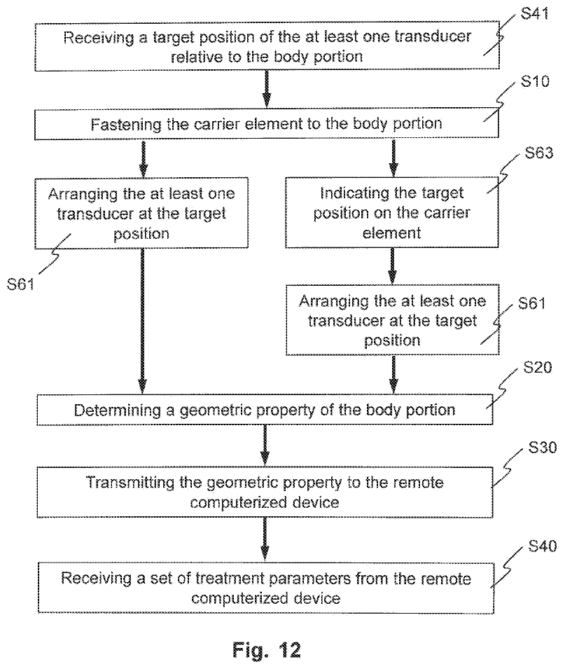

FIG. 12 shows a flow chart of an exemplary embodiment of a method for setting up a treatment device that includes an adjustment of the set of treatment parameters, in particular of the target position(s) comprised therein, wherein the adjustment considers an influence of the carrier element 1 fastened on the body portion to the body portion.

The embodiment of FIG. 12 further shows a step S63 of indicating the target positions on the carrier element 1, for example by small LEDs, said step being optional to the step S61 of arranging the transducer needed for the selected treatment at the target positions.

As a rule, the embodiment shown in FIG. 12 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIGS. 7-11 (if not present already).

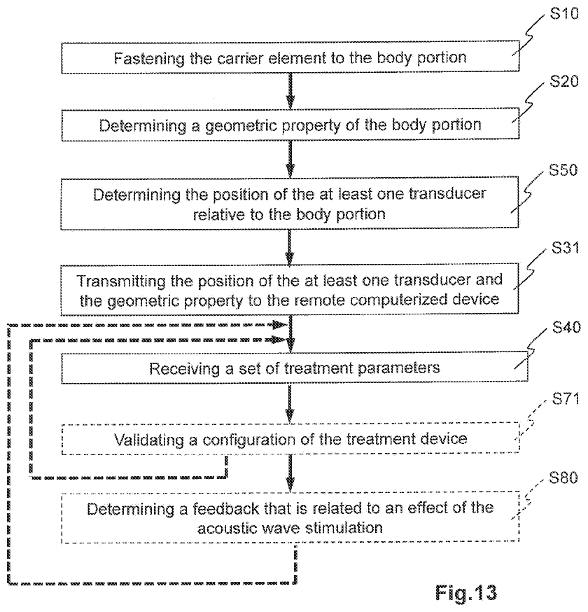

FIG. 13 shows a flow chart of an exemplary embodiment of a method for setting up a treatment device that includes an optional step S71 of validating, for example in an iterative manner, a configuration of the treatment device. The step S70 of comparing determined position(s) of the transducer(s) with target position(s) can be considered as a step S71 of validating a configuration of the treatment device. However, other parameters of the treatment device can be validated in the step S71 of validating, for example the type of transducer 2 mounted, the orientation of the transducer(s) mounted etc.

The embodiment shown in FIG. 13 includes further an optional step S80 of determining a feedback that is related to an effect of the acoustic wave stimulation. The feedback can rely on a user feedback provided by the user via the user interface 12 or it can rely on a measurement on the body portion, for example via the sensor 13 for recording subject specific information other than the geometric or acoustic properties of the body portion.

In dependence of the realization of the treatment device, in particular of the carrier element 1, the step S20 of determining a geometric property and transmission of the geometric property to the computerized device in the step S31 of transmitting the position of the transducers 2 needed and the determined geometric property to the computerized device can be omitted.

As a rule, the embodiment shown in FIG. 13 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIGS. 7-12 (if not present already).

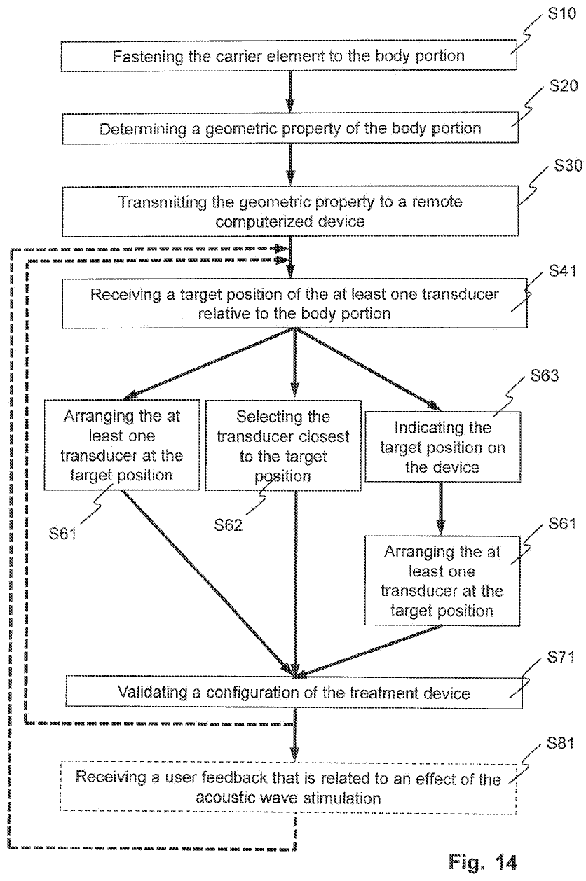

FIG. 14 shows a flow chart of another exemplary embodiment including the step S71 of validating a configuration of the treatment device and including optionally a step S81 of receiving a user feedback that is related to an effect of the acoustic wave stimulation. Said step S81 can be considered as an embodiment of the step of determining a feedback that is related to an effect of the acoustic wave stimulation.

The step S71 of validating a configuration of the treatment device is needed in the embodiment shown in FIG. 14 because the embodiment includes the step S61 of arranging the transducers needed at the target positions given by the set of treatment parameters or it includes the step S62 of selecting the transducer(s) that is/are closest to target position(s). Both steps can result in positions of the transducers 2 needed that do not correspond to the target positions in a sufficient manner. Hence, the configuration of the treatment device needs to be validated and the set of treatment parameters potentially needs to be adjusted either based on differences determined during the step S71 of validating the configuration or/or based on feedback that is a user feedback in the embodiment shown.

In dependence of the realization of the treatment device, in particular of the carrier element 1, the step S20 of determining a geometric property and the step S30 of transmitting the geometric property can be omitted.

The step S71 of validating a configuration can include the step S50 of determining the position of the needed transducers 2 relative to the body portion and the step S70 of comparing the determined positions with the target positions received in step S41.

As a rule, the embodiment shown in FIG. 14 includes further the step S1 of ready the carrier element 1, the at least one transducer 2 and the communication means 6, and the step S2 of connecting the communication means 6 to the remote computerized device as disclosed with respect to FIG. 6. It can include further the steps disclosed with respect to FIGS. 7-13 (if not present already).

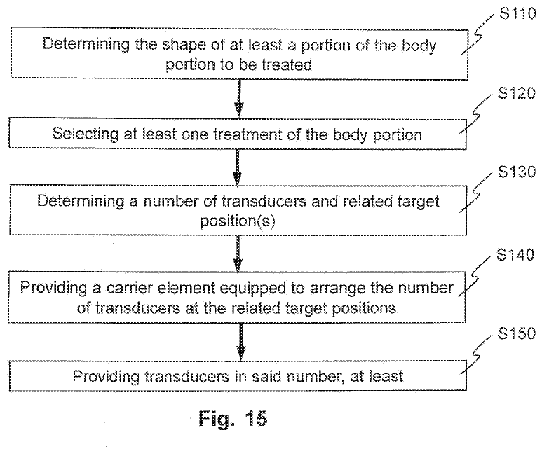

FIG. 15 shows a flow chart of an exemplary embodiment of a method for manufacturing a treatment device. The method comprises:
A step S110 of determining the shape of at least a portion of the body portion to be treated.
The shape can be determined in any manner and/or by any person disclosed above with respect to the determination of the shape of the body portion to be treated or a portion thereof.
A step S120 of selecting at least one treatment of the body portion.
In other words, the treatment device manufactured can be configured for one treatment only or for a plurality of treatments.
A step S130 of determining a number of transducers and related target position(s).
This means, that a minimum number of transducers needed for the treatment(s) selected is determined and the target position(s) needed for the selected treatment(s) is determined.
A step S140 of providing a carrier element 1 that is equipped to arrange the number of transducers at the needed target position(s).
As pointed out with respect to FIG. 6 for example, the carrier element 1 can be provided in a number of ways.
The carrier element provided may be provided in any embodiment disclosed above.
A step S150 of providing transducers in said number.
As pointed out with respect to FIG. 6 for example, the transducer can be provided in a number of ways.

As pointed out above, the method for manufacturing may include a step of assembling the transducers and the carrier element, the step being carried out by the manufacturer or provider of the carrier element 1 and the transducers 2. However, it is possible that assembling is done by a practitioner or the end-user.

1. A treatment device for acoustic wave stimulation to a body portion comprising a carrier element and at least one transducer,
wherein the treatment device is portable and in that the treatment device comprises communication means configured to receive operating parameters for the at least one transducer from a remote computerized device.

2. The device of claim 1, further comprising means for determining a geometric property of the body portion when the carrier element is fastened on the body portion, wherein a characteristic of the means for determining a geometric property depends on at least one of the shape of the carrier element and a deformation of the carrier element, wherein the treatment device is configured to transmit the geometric property of the body portion to the computerized device.

3. The device of claim 2, wherein the means for determining a geometric property of the body portion comprise means for determining a shape of the body portion to be stimulated.

4. The device of claim 2, wherein the carrier element comprises the means for determining a geometric property of the body portion.

5. The device of claim 2, wherein the means for determining a geometric property comprise at least one of
a marker arrangement, wherein the marker arrangement is arranged on the carrier element and wherein the marker arrangement changes its appearance in dependence of the shape of the carrier element;
a plurality of sensors, wherein the sensors are arranged on the carrier element and wherein an output of the sensors depends on the arrangement of the sensors on the carrier element;
a plurality of emitters, wherein the emitters are arranged on the carrier element and wherein an output of the emitters that is detectable at a detection point depends on the relative arrangement of the emitters.

6. The device of claim 1, wherein the treatment device allows for the determination of a position of the at least one transducer relative to the body portion when the carrier element is fastened on the body portion, wherein the treatment device is configured to transmit the position of the at least one transducer relative to the body portion to the computerized device.

7. The device of claim 6, wherein at least one of the following applies: the at least one transducer comprises a passive detectable element, the at least one transducer comprises an active detectable element, and the carrier element comprises at least one fixation element configured to detect the presence of a transducer.

8. The device of claim 1, wherein the carrier element has a shape that is specific to the body portion of an individual subject.

9. The device of claim 1, wherein the carrier element comprises rigid material that is formed according to at least a portion of the body portion to be treated.

10. The device of claim 1, wherein the carrier element comprises a fixation element per transducer needed for a specific application, wherein each fixation element is arranged on the carrier element at a target position for the specific application.

11. The device of claim 1, wherein the carrier element comprises an arrangement of a plurality of fixation elements that allows fixation of the at least one transducer at target positions of various applications.

12. The device of claim 1, wherein the device is configured to receive a target position of the at least one transducer from the computerized device, and wherein
the device comprises at least two transducers, wherein the device is configured to activate the transducer that is closest to the target position during stimulation, wherein the other transducer remains in an off-state; or
the carrier element comprises position marks that help to arrange the at least one transducer at the target position; or
the device is configured to determine that the at least one transducer is at the target position.

13. The device of claim 1, wherein the device is configured for validating a configuration of the device with respect to a set of treatment parameters by comparing a measured parameter of the treatment device with a related parameter of the set of treatment parameters.

14. The device of claim 1, wherein the device comprises an input element for receiving a user feedback related to an effect of the ultrasonic stimulation.

15. The device of claim 1, wherein the device comprises an input element for receiving information related to an effect of the ultrasonic stimulation, wherein the communication means are configured to transmit the information to a library that is available for other users.

16. The device of claim 1, wherein the device comprises a sensor for generating subject specific information other than the geometric property of the body portion.

17. The device of claim 1, comprising a transducer for exciting acoustic waves in the body portion and a transducer for detecting acoustic waves that are transmitted through and/or scattered in the body portion.

18. A carrier element for a treatment device according claim 1, wherein the carrier element is formed according to at least a portion of the body portion to be treated, and wherein
the carrier element comprises a fixation element for at least one transducer, wherein the number of fixation elements and the arrangement of the fixation elements are adapted to a specific acoustic wave stimulation; or
the carrier element comprises an arrangement of a plurality of fixation elements that allows fixation of at least one transducer at target positions of various acoustic wave stimulations.

19. The carrier element of claim 18, wherein the carrier element is formed according to a least a portion of the body portion of an individual subject, said body portion being the body portion to be treated.

20. The carrier element of claim 18, wherein the carrier element comprises rigid material and wherein it is the rigid material that is formed according to the portion of the body portion to be treated.

21. A system comprising a carrier element, at least one transducer, communication means and a computer program comprising instructions which, when executed by a computerized device, cause the computerized device to exchange data with the communication means.

22. A method for setting up a treatment device for acoustic wave stimulation of a body portion, wherein the device comprises at least one transducer, a carrier element and communication means, wherein the method comprises a step of receiving operating parameters for the at least one transducer from a remote computerized device.

23. The method of claim 22 comprising the further steps of:
fastening the carrier element to the body portion to be stimulated;
determining a geometric property of the body portion;
transmitting the geometric property to a computerized device capable to determine operating parameters of the treatment device.

24. The method of claim 22, wherein the method comprises a step of fastening the carrier element to the body portion to be stimulated, a step of determining a position of the at least one transducer relative to the body portion when the at least one transducer is fastened to the carrier element and the carrier element is fastened on the body portion and a step of transmitting the position of the at least one transducer to the computerized device.

25. The method of claim 24, wherein the step of determining a position of the at least one transducer relative to the body portion comprises at least one of
taking an image;
determining the presence of the at least one transducer in a fixation element of the carrier element.

26. The method of claim 24, wherein the operating parameters received are part of a set of treatment parameters received and wherein the set of treatment parameters received comprises the position of the at least one transducer as target position.

27. The method of claim 22, comprising further at least one of receiving a start signal and receiving a stop signal from the computerized device.

28. The method of claim 22, wherein the operating parameters received are part of a set of treatment parameters received.

29. The method of claim 28, wherein the set of treatment parameters received comprises a target position of the at least one transducer relative to the body portion, and wherein the method comprises a step of arranging the at least one transducer on the carrier element and at the target position.

30. The method of claim 28, wherein the device comprises a plurality of transducers, wherein the set of treatment parameters received comprises at least one target position of a transducer, and wherein the method comprises the step of activating the transducer that is closest to a target position, wherein any transducer that is not closest to a target position remains in an off-state.

31. The method of claim 28, wherein the method comprises a step of validating a configuration of the treatment device with respect to the set of treatment parameters received by comparing a measured parameter of the treatment device with a related parameter of the set of treatment parameters.

32. The method of claim 22, further comprising a step of receiving a user feedback related to an effect of the ultrasonic stimulation.

33. The method of claim 22, further comprising a step of arranging the at least one transducer on the carrier element, said step being carried out by a practitioner.

34. The method of claim 22, wherein the operating parameters are determined by a practitioner or by a computer-implemented method configured to select the operating parameters from a plurality of operating parameters that are stored in a library.

35. A method of manufacturing a portable treatment device for acoustic wave stimulation to a body portion comprising the steps of
determining the shape of at least a portion of the body portion to be treated;
selecting a treatment of the body portion;
determining a number of transducers and a target position of at least one transducer relative to the body portion, wherein the number of transducers and target positions as well as the target positions depend on the selected treatment and the shape determined;
providing a carrier element and providing transducers in said number, wherein the carrier element is equipped to arrange the transducers at the target positions.

36. The method of manufacturing of claim 35, wherein the step of determining at least a portion of the shape of the body portion, the step of selecting a treatment of the body portion and the step of determining a target position is carried out by a practitioner.

37. The method of manufacturing of claim 35, comprising a step of arranging the transducer(s) at the target position(s), said step being carried out by a practitioner.

38. The method of manufacturing of claim 35, wherein the carrier element is formed at least according to the portion of the shape of the body portion to be treated.

39. The method of manufacturing of claim 38, wherein the carrier element is formed at least according to the portion of the shape of the body portion of an individual subject to be treated.

40. The method of manufacturing of claim 35, wherein the carrier element comprises at least one portion of a rigid material, wherein the at least one portion of a rigid material forms a surface that corresponds at least to the shape of the portion of the body portion to be treated.

41. The method of manufacturing of claim 40, wherein the at least one portion of rigid material is manufactured by a rapid manufacturing process and comprises fixation elements, wherein
the fixation elements are in the number of the number of target positions and are arranged in a manner that allows fixation of the transducers at the target positions; or
the fixation elements are in a number that is larger than the number of target positions and arranged in a manner that allows fixation of the transducers at the target positions of various treatments of the body portion.
